(19) United States
(12) Patent Application Publication
(10) Pub. No.: US 2026/0237969 A1
(43) Pub. Date: Aug. 13, 2026

(54) HCG TUNABLE VCSEL WITH FULLY OXIDIZED BOTTOM DBR (GAAS BASED VCSEL)

(71) Applicant: Bandwidth10, LTD., Berkeley, CA (US)

(72) Inventors: Carlos Fernando Rondina Mateus, Berkeley, CA (US); Christopher Chase, Berkeley, CA (US); Michael C. Y. Huang, Taipei (TW); Murat Serbay, Berlin (DE); Stefano Prandoni, Berlin (DE); Philip Worland, Berkeley, CA (US)

(21) Appl. No.: 19/430,134

(22) Filed: Dec. 22, 2025

Related U.S. Application Data
(63) Continuation-in-part of application No. 18/223,535, filed on Jul. 18, 2023, now Pat. No. 12,502,104.
Application No. 18/223,535 is a continuation-in-part of application No. 18/107,140, filed on Feb. 8, 2023, now Pat. No. 12,224,559.
Application No. 18/107,140 is a continuation-in-part of application No. 18/111,902, filed on Feb. 21, 2023.
Application No. 18/111,902 is a continuation-in-part of application No. 18/207,804, filed on Jun. 9, 2023, now Pat. No. 12,301,291.
Application No. 18/207,804 is a continuation-in-part of application No. 18/207,810, filed on Jun. 9, 2023, now Pat. No. 12,294,199.
Application No. 18/207,810 is a continuation-in-part of application No. 18/207,818, filed on Jun. 9, 2023, now Pat. No. 12,316,075.
Application No. 18/207,818 is a continuation-in-part of application No. 18/207,826, filed on Jun. 9, 2023, now Pat. No. 12,300,970.
Application No. 18/207,826 is a continuation-in-part of application No. 18/207,864, filed on Jun. 9, 2023, now abandoned.
Application No. 18/207,864 is a continuation-in-part of application No. 18/207,838, filed on Jun. 9, 2023, now Pat. No. 12,278,462.
Application No. 18/207,838 is a continuation-in-part of application No. 18/207,857, filed on Jun. 9, 2023, now Pat. No. 12,298,436.
Application No. 18/207,857 is a continuation-in-part of application No. 18/214,986, filed on Jun. 27, 2023, now Pat. No. 12,514,472.
Application No. 18/214,986 is a continuation-in-part of application No. 18/215,200, filed on Jun. 28, 2023, now abandoned.
(60) Provisional application No. 63/402,546, filed on Aug. 31, 2022.
(60) Provisional application No. 63/402,553, filed on Aug. 31, 2022.
(60) Provisional application No. 63/402,556, filed on Aug. 31, 2022.
(60) Provisional application No. 63/402,560, filed on Aug. 31, 2022.
(60) Provisional application No. 63/415,268, filed on Oct. 11, 2022.
(60) Provisional application No. 63/432,050, filed on Dec. 12, 2022.

(51) Int. Cl. H01S 5/183; H01S 5/30; H01S 5/343

(52) U.S. Cl. CPC H01S 5/18311; H01S 5/18361; H01S 5/18394; H01S 5/3095; H01S 5/343

(57) ABSTRACT

A light emitting apparatus includes a VCSEL system with a tunable VCSEL laser having a first mirror; one or more active regions with a first active region adjacent to the first mirror, each of an active region including quantum wells and barriers, each of an active region surrounded by one or more p-n junctions, the one or more active regions including a selected shape structure each with a tunnel junction (TJ) of a plurality of TJ's; one or more apertures provided with the selected shape structure; one or more buried tunnel junctions (BTJ), additional TJ's, planar structures and a plurality of additional BTJ's are created during a regrowth process that is independent of a first growth process of the first mirror, the active region and the one or more TJs; one or more oxide layers and an epitaxial growth of an AlGaAs; one or more electrical confinement apertures defined by the one or more BTJ's, additional TJ's, planar structures and additional BTJ; a vertical resonator cavity disposed over the electrical confinement aperture; a high contrast grating (HCG) operating as a second mirror positioned over the vertical resonator cavity, the HCG configured to reflect a first portion of light back into the vertical resonator cavity, and a second portion of the light as an output beam from the tunable VCSEL laser, the HCG structure being layered on the selected shape structure. A shape of the output beam of the tunable VCSEL laser is determined by a geometric shape of the one or more BTJ apertures, apertures for additional TJ's, planar structures and additional BTJ's, with a transmission function of the HCG and is designed according to the desired optical transmission function of the application. A DBR is on one side of the junctions, and an HCG grading on the other side of the junctions. The VCSEL has a final thickness of an oxide layer different than the epitaxial growth of the AlGaAs.

### CROSS-REFERENCE TO RELATED APPLICATIONS

This patent application is continuation in part of U.S. Non-Provisional patent application Ser. No. 18/223,535, title ‘HEALTH-MONITORING DEVICE WIT VCSEL’, filed on Jul. 18, 2023, which claims priority from U.S. Provisional Application No. 63/402,546, title ‘HCG TUNABLE VCSEL WITH FULLY OXIDIZED BOTTOM DBR (GAAS BASED VCSEL)’, filed on Aug. 31, 2022; U.S. Provisional Application No. 63/402,553, title ‘HCG TUNABLE VCSEL WITH OPTICAL CONFINEMENT VIA STEP INDEX AND REGROWTH’, filed on Aug. 31, 2022, U.S. Provisional Application No. 63/402,556, title ‘HCG TUNABLE VCSEL WITH ELECTRICAL AND OPTICAL CONFINEMENT VIA ETCHED POST’, filed on Aug. 31,2022; U.S. Provisional Application No. 63/402,560, title ‘FLIP CHIP TUNABLE VCSEL WITH HCG ON SI SUBMOUNT’, filed on Aug. 31, 2022; U.S. Provisional Application No. 63/415,268, title ‘LIGHT EMITTING DEVICE WITH DETEMINABLE SHAPE OF OUTPUT BEAM’, filed on Oct. 11, 2022 and U.S. Provisional Application No. 63/432,050, title ‘HCG TUNABLE VCSEL WITH INTEGRATED DETECTOR IN THE SACRIFICIAL LAYER’, filed on Dec. 12, 2022; is a continuation in part of U.S. Non-Provisional patent application Ser. No. 18/107,140, title ‘LIGHT EMITTING DEVICE WITH DETERMINABLE SHAPE OF OUTPUT BEAM’, filed on Feb. 8, 2023; U.S. Non-Provisional patent application Ser. No. 18/111,902, title ‘HCG TUNABLE VCSEL WITH ELECTRICAL AND OPTICAL CONFINEMENT VIA ETCHED POST’, filed on Feb. 21, 2023; U.S. Non-Provisional patent application Ser. No. 18/207,804, title ‘SYSTEMS FOR DETECTING GESTURES’, filed on Jun. 9, 2023; U.S. Non-Provisional patent application Ser. No. 18/207,810, title ‘SYSTEMS USING (3D) MAPS OF AT LEAST A PART OF A BODY OF A USER’, filed on Jun. 9, 2023; U.S. Non-Provisional patent application Ser. No. 18/207,818, title ‘SYSTEMS INCLUDING ONE OR MORE VCSELS, FOR RECEIVING A THREE DIMENSIONAL (3D) MAP OF AT LEAST A PART OF A BODY OF A USER’, filed on Jun. 9, 2023; U.S. Non-Provisional patent application Ser. No. 18/207,826, title ‘DIGITAL CAMERA WITH VCSELS”, filed on Jun. 9, 2023; U.S. Non-Provisional patent application Ser. No. 18/207,864, title ‘OPTOELECTRONIC DEVICE WITH VCSEL PROVIDING A GRID PATTERN”, filed on Jun. 9, 2023; U.S. Non-Provisional patent application Ser. No. 18/207,838, title ‘APPARATUS FOR FACIAL RECOGNITION”, filed on Jun. 9, 2023; U.S. Non-Provisional patent application Ser. No. 18/207,857, title ‘DEPTH MAPPING SENSING APPARATUS”, filed on Jun. 9, 2023; U.S. Non-Provisional patent application Ser. No. 18/214,986, title ‘HEALTH-MONITORING MEASUREMENT AND/OR MONITORING WITH A SMARTWATCH THAT INCLUDES ONE OR MORE VCSEL'S”, filed on Jun. 27, 2023; U.S. Non-Provisional patent application Ser. No. 18/215,200, titled ‘VCSEL WITH TUNABLE GRATING”, filed on Jun. 28, 2023.

### FIELD OF THE INVENTION

This invention relates to VCSEL's, and more particularly to an HCG tunable VCSEL with a fully oxidized bottom DBR (GAAS based vcsel), and its methods of manufacture.

### DESCRIPTION OF THE RELATED ART

Mirror structures suitable for use in resonant cavity devices, such as vertical cavity surface emitting lasers, have been disclosed, see U.S. No. 7,251,264, incorporated herein by reference.

Selective oxidation has been used for definition of electrical aperture. Full selective oxidation of bottom DBR has been disclosed in addition to electrical aperture oxidation, an n-doped gallium arsenide (GaAs) substrate has an n-type electrical contact. An n-doped lower mirror stack (a DBR) is positioned on the GaAs substrate, and an n-type lower spacer is disposed over the lower mirror stack. An active region, usually having a number of quantum wells, is formed over the lower spacer. A p-type top spacer is disposed over the active region, and a p-type top mirror stack (another DBR) is positioned over the top spacer. A p-type conduction layer, a p-type GaAs cap layer, and a p-type electrical contact are over the top mirror stack. An oxide aperture forming layer has a relatively high Al content (e.g., over 95%, and beneficially about 98%) to facilitate lateral oxidation, trenches or holes can be etched around at least part of a periphery of the VCSEL to facilitate the lateral oxidation of the oxide aperture forming layer.

Geske, Bowers, Leonard and MacDougal demonstrated record wavelength span from an 8-channel WDM VCSEL array operating CW to 65° C. from 1470 nm to 1610 nm with precise 20-nm channel spacing. The devices were fabricated using nonplanar wafer bonding and fully-oxidized DBRs. Precise 20-nm wavelength spacing and single transverse mode operation is maintained for all channels. These devices were optically pumped using a fiber coupled 980-nm pump laser, a technique compatible with long-wavelength VCSELs. This result was possible by the simultaneous application of a simplified nonplanar wafer bonding procedure and through the use of broadband fully oxidized GaAs/AlOx DBRs

Geske and Bowers, Appl. Phys. Lett., vol. 79(2001 ). Pp. 1760-1762, have disclosed methods of nonplanar wafer bonding, that is capable of simultaneously integrating multiple active regions across a wafer surface in a single wafer bonding step. This has been used to fabricate long-wavelength arrays with a narrow wavelength span using traditional AlGaAs distributed Bragg reflectors (DBRs).

Geske, Okuno and Bowers, IEEE Photon Technol. Lett., vol 15 (2003), pp. 179-181, have disclosed fabrication of a dual-wavelength 1550 nm and 1530 nm VCSEL array using two separate and distinct active regions integrated on a common mirror by nonplanar wafer bonding. The nonplanar wafer bond used in the fabrication device begins with two strained multi-quantum-well long-wavelength VCSEL active regions grown vertically on an InP wafer and separated by an etch-stop layer. The active regions have optical cavity lengths of 2.5 wavelengths at 1550 and 1530 nm and have photoluminescence peaks at 1530 nm. The wafer surface is etched with a step shaped profile to reveal a different active region on each step level. The backside of the wafer is etched to have a profile complimentary to the step etched active region side of the wafer. The completed VCSEL array has lasing wavelengths of 1550 and 1530nm.

Gunwu et al., Optoelectronics and Advanced Materials-Rapid Communications, Vol. 12, 3-4, March-April 2018. P. 137-139, have disclosed uniform and reproducible high-index-contrast distributed Bragg reflectors (HIC-DBRs) by introducing an oxidized digital-alloy (DA) layer for low index materials. Fine-tunable DA-Al0.98Ga0.02As layers, which consists of multiple stacks of 1 monolayer (ML)-thick GaAs and 49 ML-thick AlAs layers, are used for generating stable Al xOy layers instead of a conventional, bulk AlAs layers. Highly uniform arrays of oxidized alumina are obtained through a wet-oxidation of AlGaAs layers, which enable practical use of HIC-DBRs for vertical cavity surface emitting lasers (VCSELs) and other resonant-cavity based optoelectronic devices. Detailed experiments together with optical calculations are also shown. Wet-oxidized pure AlAs/GaAs DBR and DA-AlGaAs/GaAs DBR were designed. The DA-Al0.98Ga0.02As layer consisted of 12 pairs of 1 monolayer (ML)-thick GaAs and 49 ML-thick AlAs layers. The thicknesses were designed for 980 nm center wavelength. The aluminum oxide thickness was designed to be thicker than quarter wavelength thickness considering 12% shrinkage. All structures were grown on semi-insulating GaAs substrates by molecular beam epitaxy (MBE) system. The growth rate of GaAs and AlAs materials were measured by RHEED intensity oscillation before growth sequence. For lateral oxidation of the samples, 110 μm diameter cylinder mesa patterns were etched by inductively coupled plasma reactive ion etching (ICP-RIE). The etched samples were immediately oxidized in a tube furnace at 420 degrees C under 2 l/min flow of nitrogen carrier which is bubbled through 90 degree C deionized water reservoir. The oxidation was processed with different oxidation times to ascertain the oxidation rates.

The rapid oxidation rate of the pure AlAs layer resulted in non-uniform oxidation characteristic. After a selected oxidation time, the pure AlAs showed non-uniform oxidation characteristics, while the DA-AlGaAs sample demonstrated uniformly oxidized in spite of a relatively slow oxidation rate. The oxidation rate of the DA-DBR layer was faster than the aperture because the thickness of the DA-DBR layer is thicker than the aperture thickness. The DA-DBR was completely oxidized before the aperture closed.

A model of the thermal oxidation kinetics that is based on the continuity equation is adapted to the fabrication of circular saturable Bragg reflector (SBR) mesa structures, as well as inverted mesa array structures.

A semiconductor Saturable Bragg Reflector (SBR) is a mirror structure comprising alternating layers of high and low refractive index materials with an incorporated saturable absorber. SBRs can be used to initiate and sustain ultra-short pulses in various laser systems. In order to form ultra-short pulses, SBRs with high reflectivity over a broad wavelength range are required. Furthermore, large-area SBRs facilitate easy integration in a laser cavity. One of the key elements for the realization of broad band SBRs is the development of the thermal oxidation process that creates buried low-index AlxOy layers over large areas. The design, fabrication, characterization, and implementation of broad band, high index contrast III-V/AlxOy SBRs in the form of circular mesas, as well as inverted mesa structures, see Nabanja et al.

A model of the thermal oxidation kinetics that is based on the continuity equation is adapted to the fabrication of circular saturable Bragg reflector (SBR) mesa structures, as well as inverted mesa array structures. A comparison between theoretical calculations and experimental measurements shows good agreement, making the model a useful tool for the controllability of the structure-dependent oxidation process. The model is applied to the wet oxidation of buried AlAs layers for the fabrication of ultra-broadband, high index contrast, III-V/AlxOy SBRs. The rate of oxidation is found to be dependent not only on the oxidation temperature, but also on oxidation time, mesa geometry, and AlAs layer thickness. The two distinct large-area SBR structures, the circular mesa and the inverted mesa, were fabricated.

A buried oxide grating structure has been formed by selectively-oxidized AlxGa1-xAs grown on nonplanar substrates uses low-pressure MOCVD. Localized aluminum content variation in AlGaAs is obtained with MOCVD growth on nonplanar substrate. Buried aluminum oxide/semiconductor distributed feedback structure was achieved with selective oxidation of these AlGaAs layers, see Nabanja et al., Journal of Applied Physics 113, 163102 (2013).

Wu et al., Electron. Let. 33. 1702-1703 (1997), has disclosed a widely tunable 1.5 um micromechanical optical filter using a AlOx/AlGaAs DBR.

Jayaraman et al., The International Society for Optical Engineering, 2012.8276:82760D,11; Electron Lett. 2012:48(14): 867-869, disclose 1060 nm. microelectromechanical-systems-based tunable vertical-cavity surface-emitting lasers (MEMS-VCSELs) with a 100 nm continuous tuning range under repetitively scanned operation at rates beyond 500 kHz and a 90 nm continuous tuning range under static operation. These devices employ a thin strained InGaAs multiple quantum well active region integrated with a fully oxidized GaAs/AlxOy bottom mirror and a suspended dielectric top mirror. The devices are optically pumped via 850 nm light. The ultra-widely tunable lasers represent the first MEMS-VCSELs reported in this wavelength range, and are ideally suited for application in ophthalmic swept-source optical coherence tomography.

MacDougal, et al., IEEE Photonics Technology Letters, Vol. 7. No 3. March 1995, has disclosed ultralow threshold current vertical-cavity surface-emitting lasers obtained with selective oxidation. InGaAs single quantum well vertical-cavity surface-emitting lasers were produced with an intracavity p-contact fabricated by selective oxidation of AlAs and distributed Bragg reflectors composed of binary materials (AlAs/GaAs). Low threshold currents of 8.7 μA in ~3 μm square devices and 140 μA in 10 μm square devices with maximum output powers over 1.2 mW were achieved.

John et al., Journal of Lightwave Technology, Feb. 2, 2015, 33(16): 3461-3468, has disclosed that the wide wavelength tuning range, rapid tuning rate, and long coherence length of vertical cavity surface emitting lasers with micro-electromechanical systems-based tuning mechanisms (MEMS-VCSELs) has made these optical sources an attractive technology for swept-source optical coherence tomography (SS-OCT). Optically pumped MEMS-VCSEL light sources have been demonstrated in SS-OCT systems with variable imaging rates up to 1 MHz, axial resolution of 9 μm, record centimeter range imaging of large objects, full eye-length measurements, optical fiber measurement greater than 1 m, and hand-held OCT. SS-OCT used in ophthalmology can prevent irreversible vision-loss by enabling early detection and monitoring of ocular disease in a non-invasive manner. In ophthalmology, the long coherence length combined with the variable and high-speed imaging capabilities of the previously described optically pumped 1050 nm MEMS-VCSEL has enabled new OCT capability, such as multipurpose instrumentation that can image the retina and anterior eye, and perform full eye length measurement, enface Doppler blood flow measurement techniques with rapidly repeated volumes to visualize pulsatility, and densely sampled dye-free OCT angiography (OCTA).

Transitioning from an optically-pumped MEMS-VCSEL system as in to direct electrical injection would remove the need for a separate pump laser, pump laser drivers/temperature controllers, and the dichroic optical splitter, which dramatically reduces the size and cost of the light source.

Although electrically-pumped MEMS-VCSELs at 1550 nm have been previously developed, the first electrically-pumped MEMS-VCSEL at 1050 nm, suitable for ophthalmic OCT, was demonstrated by Ansbaek et al. in 2013 with 24 nm of tuning using a high-contrast grating mirror.

In one embodiment, the lasing cavity, is comprised of a movable top mirror, air gap, multiple quantum wells (MQW) gain region, and finally a fully oxidized back mirror. The top mirror, a wide-band distributed Bragg reflector (DBR), is suspended over an air gap that is electrostatically adjustable via an applied tuning voltage across the top and bottom actuator contacts. Electrostatic contraction of the air gap consequently decreases the resonance wavelength of the Fabry-Pérot cavity mode, and thus the lasing wavelength

Choquette al el., IEEE. J. Sel. Top. Quantum Electron. 3, 916 (1997 ), has disclosed selective conversion of buried layers of AlGaAs to a stable oxide and the implementation of this oxide, into high performance vertical-cavity surface emitting lasers (VCSELs). The rate of lateral oxidation was shown to be linear with an Arrhenius temperature dependence. The measured activation energies varied with A1 composition, providing a high degree of oxidation selectivity between AlGaAs alloys. buried oxide layers can be selectively fabricated within the VCSEL through small compositional variations in the AlGaAs layers. The oxidation of AlGaAs alloys, as opposed to AlAs, provides robust processing of reliable lasers. The insulating and low refractive index oxide provides enhanced electrical and optical confinement for ultralow threshold currents in oxide-apertured VCSELs.

In Choquette et al, an elective oxidation of high Al content layers is well known and largely used for manufacturing of VCSELs. The high index of refraction difference provided in between oxidized and non-oxidized material does suggest selective oxidation as a direct candidate for a DBR mirror. However, process to develop this selective oxidation of the bottom DBR on a monolithically tunable VCSEL (no bonding, no regrowth) with HCG type of mirror is not obvious. Moreover, electrically pumped VCSELs require a path of conductive semiconductor material in order to allow electrical current to reach the active region. Thus, design of semiconductor layers has to be carefully done to account for several parameters, including controlled shrinkage of oxidized layers and change on index of refraction. Final design is non-obvious for experts and manufacturing requires integration and compatibility of several different processes, not required for the conventional semiconductor as-grown DBR.

In the formation of Al₂O₃, as disclosed by Choquette et al. the oxidation state of Al is made more positive by oxidation, and thus the oxidation state of another constituent in the reaction must become more negative, i.e., it must be reduced. In dry oxidation, this is accomplished by the change from zero-valent O₂or O atoms to O⁻².

However, for a wet oxidation process that uses steam in an inert carrier gas, the oxygen in water is already in the two-oxidation state. Consequently, the [symbol] for removing electrons (the oxidizing agent) from AlAs to produce Al₂O₃ is H from water. Moreover, as discussed below, the resulting zero-valent H can produce volatile as species by reduction of the intermediate As₂O₃.

Choquette et al. has shown that hydrogen has a central role in the oxidation reaction, serving as the oxidizing species to form Al₂O₃ and As₂O₃ as well as reduction of As₂O₃ to As. This explains the orders-of-magnitude faster oxidation of high Al-content semiconductors with water in an oxygen-free carrier gas than with pure oxygen. The oxide converted from AlGaAs is found to be an amorphous solid solution of (AlₓGa_{(1-x) 2}O_{c}. For buried oxide layers, no intrinsic defects or voids are detected at the oxide/semiconductor interface, although a decrease in the layer volume upon oxidation can produce strain fields around the oxide terminus. Reproducible and linear oxidation rates can be achieved by control of the gas flow.

Choquette et al. report that further work is required to develop selective wet oxidation of AlGaAs into a manufacturable technology.

However, Choquette et al. fails to disclose the method or process of using oxidation for the formation of a VCSEL with a VCSEL, and its methods of manufacture, with a fully oxidized bottom DBR, that provides a broader stop band mirror for an increased tuning range.

Yang et al., Opt. Mater. Express 1, 451, (2011), has disclosed vertical-cavity surface-emitting lasers (VCSELs) fabricated using selective oxidation to form a current aperture and an oxide surface barrier to seal buried AlAs layers in distributed Bragg reflectors. The lateral selective oxidation of the AlAs layer is strongly influenced by its thickness and heterointerface structures. The oxidation rate decreases rapidly with decreasing AlAs thickness in the range of <80 nm, and the presence of AlGaAs layers on both sides of the AlAs layer reduces the oxidation rate. In addition, an oxide surface barrier with a thickness of 1 μmis formed by a brief wet oxidation which blocks diffusing oxygen species during the second wet oxidation. The effectiveness of the seal is demonstrated by using the oxide barrier as a mask against wet oxidation in the fabrication of oxide-confined VCSELs. The 8×8 μm² devices with three quantum wells exhibit a threshold current of 400 μA, and the 4×4 μm² single-quantum devices possess a threshold current as low as 85 μA.

There is a need for an improved VCSEL

### SUMMARY

An object of the present invention is to provide a VCSE, and its methods of manufacture, with a fully oxidized bottom DBR.

Another object of the present invention is to provide a 1050 nm bandwidth VCSEL, and its methods of manufacture, with a fully oxidized bottom DBR.

A further object of the present invention, is to provide a VCSEL, and its methods of manufacture, with a with fully oxidized bottom DBR, that provides a broader stop band mirror for an increased tuning range.

Yet another object of the present invention is to provide a VCSEL, and its methods of manufacture, to obtain a broader mirror stop band in order to increase tuning range. These and other objects of the present invention are achieved in a light emitting apparatus including a VCSEL system with a tunable VCSEL laser having a first mirror; one or more active regions with a first active region adjacent to the first mirror, each of an active region including quantum wells and barriers, each of an active region surrounded by one or more p-n junctions, the one or more active regions including a selected shape structure each with a tunnel junction (TJ) of a plurality of TJ's; one or more apertures provided with the selected shape structure; one or more buried tunnel junctions (BTJ), additional TJ's, planar structures and a plurality of additional BTJ's are created during a regrowth process that is independent of a first growth process of the first mirror, the active region and the one or more TJs; one or more oxide layers and an epitaxial growth of an AlGaAs; one or more electrical confinement apertures defined by the one or more BTJ's, additional TJ's, planar structures and additional BTJ; a vertical resonator cavity disposed over the electrical confinement aperture; a high contrast grating (HCG) operating as a second mirror positioned over the vertical resonator cavity, the HCG configured to reflect a first portion of light back into the vertical resonator cavity, and a second portion of the light as an output beam from the tunable VCSEL laser, the HCG structure being layered on the selected shape structure. A shape of the output beam of the tunable VCSEL laser is determined by a geometric shape of the one or more BTJ apertures, apertures for additional TJ's, planar structures and additional BTJ's, with a transmission function of the HCG and is designed according to the desired optical transmission function of the application. A DBR is on one side of the junctions, and an HCG grading on the other side of the junctions. The VCSEL has a final thickness of an oxide layer different than the epitaxial growth of the AlGaAs.

### BRIEF DESCRIPTION OF DRAWINGS

FIGS. 1A-1D illustrate embodiments of a light emitting device of the present invention.

FIG. 2 illustrates an embodiment with several stepped BTJ apertures with different geometric shapes.

FIG. 3 illustrates a shaped structure, which can be a plateau, of the present invention.

FIG. 4 illustrates one embodiment of a P-N junction.

FIG. 5 illustrates an embodiment of FIG. 1A with an output beam and an optical beam inside of a cavity.

FIG. 6 illustrates one embodiment of electrical current confinement through a BTJ aperture, where electrical current flow from a top to a bottom contact.

FIG. 7 is similar to FIG. 3, but with a planarized HCG final layer.

FIG. 8 illustrates one embodiment of a manufacturing sequence with a growth of a bottom, a timed etch of the TJ, leaving some p++, and a regrowth on top of a VCSEL from an etched seed layer.

FIG. 9 illustrates one embodiment of a manufacturing sequence with a timed slow etch.

FIG. 10 illustrates one embodiment of a manufacturing sequence with a regrowth of n-InP with grading, and a regrowth of a top DBR, sacrificial layer and HCG layer.

FIGS. 11A and 11B illustrate a first regrowth including a bottom DBR, active layer (region), top DBR and other spacing layers.

FIGS. 12A and 12B illustrates one embodiment of implantation.

FIGS. 13A and 13B illustrate cross sections and top views of a mesa etch of the present invention.

FIGS. 14A and 14B illustrate cross sections and top views of ALD wall protection for further selective etch on release, reliability, of the present invention.

FIGS. 15A and 15B illustrate cross sections and top views of InP growth of the present invention.

FIGS. 16A and 16B illustrate cross sections and top views of the HCG layer of the present invention.

FIGS. 17A and 17B illustrate cross sections and top views of the HCG/MEMS litho/dry Etch of the present invention.

FIGS. 18A and 18B illustrate cross sections and top views of the MO-MEMs anchors, of the present invention.

FIGS. 19A and 19B illustrate cross sections and top views of the second mesa etch, M1 access of the present invention.

FIGS. 20B and 20A illustrate cross sections and top views of the second mesa etch, M1 deposit, of the present invention.

FIGS. 21B and 21A illustrate cross sections and top views of the M1 cut of the present invention.

FIGS. 22A and 22B illustrate cross sections and top views of the M1 cut of the present invention.

FIGS. 23A and 23B illustrate one embodiment of a semiconductor optical amplifier of the present invention.

FIGS. 24-27 illustrate various embodiments for the use of artificial intelligence (AI) with VCSEL amplifiers of the present invention.

### DETAILED DESCRIPTION

As used herein, the term engine refers to software, firmware, hardware, or other component that can be used to effectuate a purpose. The engine will typically include software instructions that are stored in non-volatile memory (also referred to as secondary memory) and a processor with instructions to execute the software. When the software instructions are executed, at least a subset of the software instructions can be loaded into memory (also referred to as primary memory) by a processor. The processor then executes the software instructions in memory. The processor may be a shared processor, a dedicated processor, or a combination of shared or dedicated processors. A typical program will include calls to hardware components (such as I/O devices), which typically requires the execution of drivers. The drivers may or may not be considered part of the engine, but the distinction is not critical.

As used herein, the term database is used broadly to include any known or convenient means for storing data, whether centralized or distributed, relational or otherwise.

As used herein a mobile device includes, but is not limited to, a cell phone, such as Apple's iPhone®, other portable electronic devices, such as Apple's iPod Touches®, Apple's iPads®, and mobile devices based on Google's Android® operating system, and any other portable electronic device that includes software, firmware, hardware, or a combination thereof that is capable of at least receiving a wireless signal, decoding if needed, and exchanging information with a server. Typical components of mobile device may include but are not limited to persistent memories like flash ROM, random access memory like SRAM, a camera, a battery, LCD driver, a display, a cellular antenna, a speaker, a BLUETOOTH® circuit, and WIFI circuitry, where the persistent memory may contain programs, applications, and/or an operating system for the mobile device. For purposes of this application, a mobile device is also defined to include a fob, and its equivalents.

As used herein, the term “computer” is a general-purpose device that can be programmed to carry out a finite set of arithmetic or logical operations. Since a sequence of operations can be readily changed, the communication device can solve more than one kind of problem. A communication device can include of at least one processing element, typically a central processing unit (CPU) and some form of memory. The processing element carries out arithmetic and logic operations, and a sequencing and control unit that can change the order of operations based on stored information. Peripheral devices allow information to be retrieved from an external source, and the result of operations saved and retrieved. Communication device also includes a graphic display medium.

As used herein, the term “internet” is a global system of interconnected computer networks that use the standard Network Systems protocol suite (TCP/IP) to serve billions of users worldwide. It is a network of networks that consists of millions of private, public, academic, business, and government networks, of local to global scope, that are linked by a broad array of electronic, wireless and optical networking technologies. The internet carries an extensive range of information resources and services, such as the inter-linked hypertext documents of the World Wide Web (WWW) and the infrastructure to support email. The communications infrastructure of the internet consists of its hardware components and a system of software layers that control various aspects of the architecture.

As used herein, the term “extranet” is a computer network that allows controlled access from the outside. An extranet can be an extension of an organization's intranet that is extended to users outside the organization in isolation from all other internet users. An extranet can be an intranet mapped onto the public internet or some other transmission system not accessible to the general public, but managed by more than one company's administrator(s). Examples of extranet-style networks include but are not limited to:

LANs or WANs belonging to multiple organizations and interconnected and accessed using remote dial-up

LANs or WANs belonging to multiple organizations and interconnected and accessed using dedicated lines

Virtual private network (VPN) that is comprised of LANs or WANs belonging to multiple organizations, and that extends usage to remote users using special “tunneling” software that creates a secure, usually encrypted network connection over public lines, sometimes vian ISP.

As used herein, the term “Intranet” is a network that is owned by a single organization that controls its security policies and network management. Examples of intranets include but are not limited to: s LAN, a Wide-area network (WAN) that is comprised of a LAN that extends usage to remote employees with dial-up access, a WAN that is comprised of interconnected LANs using dedicated communication lines, a Virtual private network (VPN) that is comprised of a LAN or WAN that extends usage to remote employees or networks using special “tunneling” software that creates a secure, usually encrypted connection over public lines, sometimes vian Internet Service Provider (ISP).

For purposes of the present invention, the Internet, extranets and intranets collectively are referred to as (“Network Systems”).

As used herein “Cloud Application” refers to cloud application services or “software as a service” (SaaS) which deliver software over the Network Systems eliminating the need to install and run the application on a device.

As used herein “Cloud Platform” refers to a cloud platform services or “platform as a service” (PaaS) which deliver a computing platform and/or solution stack as a service, and facilitates the deployment of applications without the cost and complexity of obtaining and managing the underlying hardware and software layers.

As used herein “Cloud System” refers to cloud infrastructure services or “infrastructure as a service” (IAAS) which deliver computer infrastructure as a service with raw block storage and networking.

As used herein “Server” refers to server layers that consist of computer hardware and/or software products specifically designed for the delivery of cloud services.

As used herein, the term “user monitoring”: (i) cardiac monitoring, which generally refers to continuous electrocardiography with assessment of the user's condition relative to their cardiac rhythm. A small monitor worn by an ambulatory user for this purpose is known as a Holter Monitor. Cardiac monitoring can also involve cardiac output monitoring vian invasive Swan-Ganz catheter (ii) hemodynamic monitoring, which monitors the blood pressure and blood flow within the circulatory system. Blood pressure can be measured either invasively through an inserted blood pressure transducer assembly, or noninvasively with an inflatable blood pressure cuff. (iii) respiratory monitoring, such as: pulse oximetry which involves measurement of the saturated percentage of oxygen in the blood, referred to as SpO2, and measured by an infrared finger cuff, capnography, which involves CO2 measurements, referred to as EtCO2 or end-tidal carbon dioxide concentration. The respiratory rate monitored as such is called AWRR or airway respiratory rate). (iv) respiratory rate monitoring through a thoracic transducer belt, an ECG channel or via capnography, (v) Neurological monitoring, such as of intracranial pressure. Special user monitors can incorporate the monitoring of brain waves electroencephalography, gas anesthetic concentrations, bispectral index (BIS), and the like, (vi) blood glucose monitoring using glucose sensors. (vii) childbirth monitoring with sensors that monitor various aspects of childbirth. (viii) body temperature monitoring which in one embodiment is through an adhesive pad containing a thermoelectric transducer. (ix) stress monitoring that can utilize sensors to provide warnings when stress levels signs are rising before a human can notice it and provide alerts and suggestions. (x) epilepsy monitoring. (xi) toxicity monitoring, (xii) general lifestyle parameters, (xiii) sleep, including but not limited to: sleep patterns, type of sleep, sleep disorders, movement during sleep, waking up, falling asleep, problems with sleep, habits during, before and after sleep, time of sleep, length sleep in terms of the amount of time for each sleep, body activities during sleep, brain patterns during sleep and the like (xiv) body gesture, movement and motion (xv) body habits, (xvi) and the like.

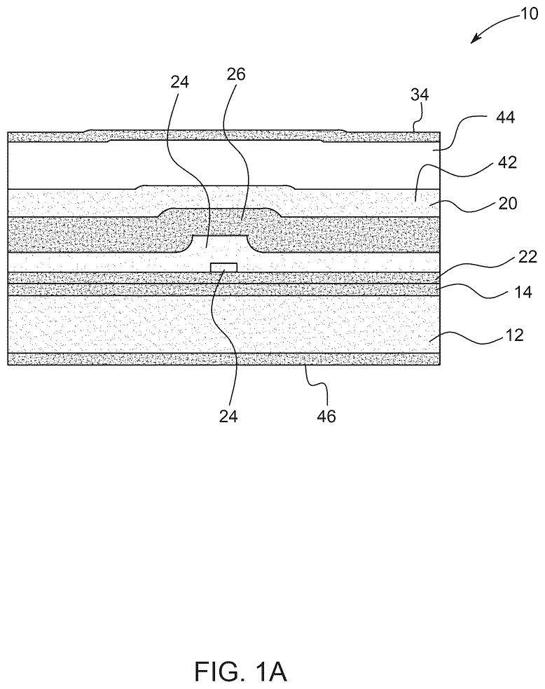

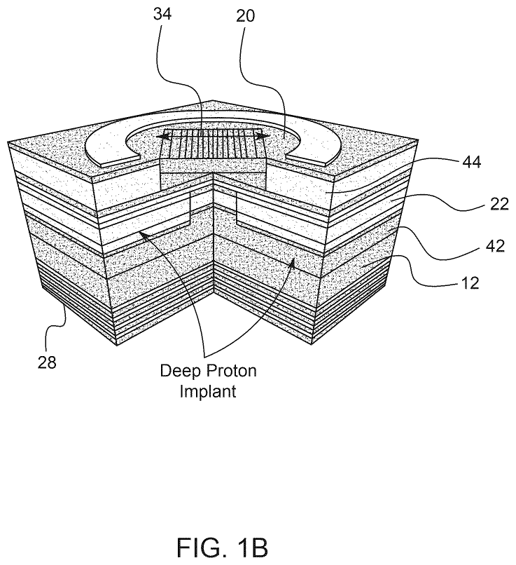

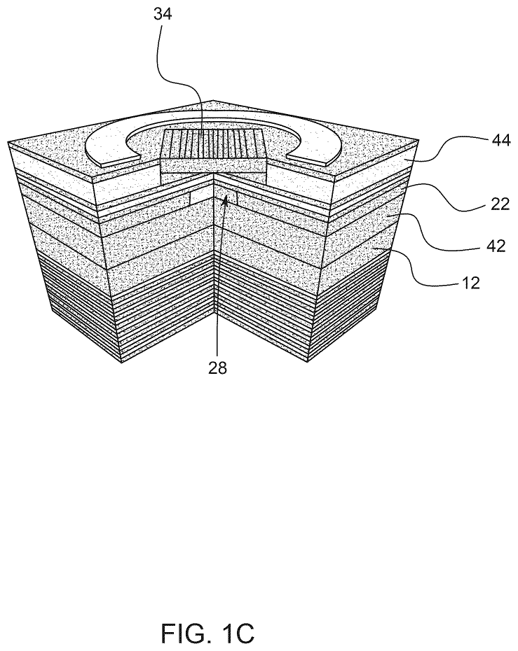

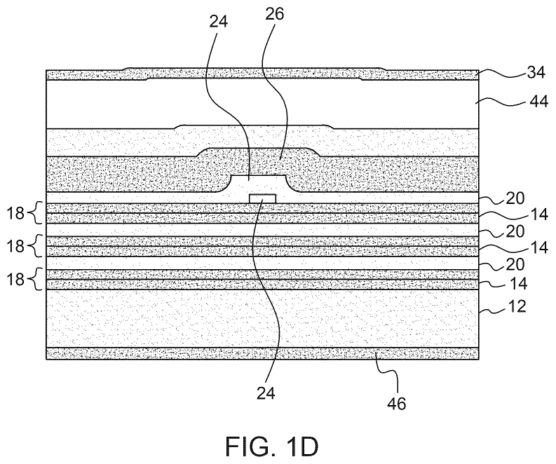

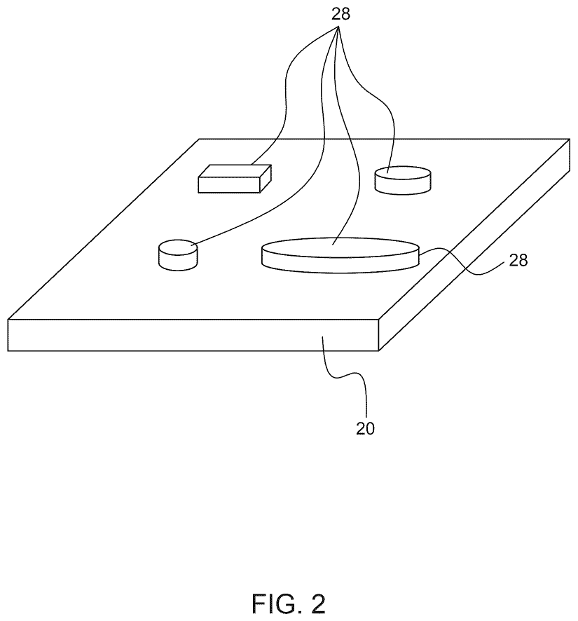

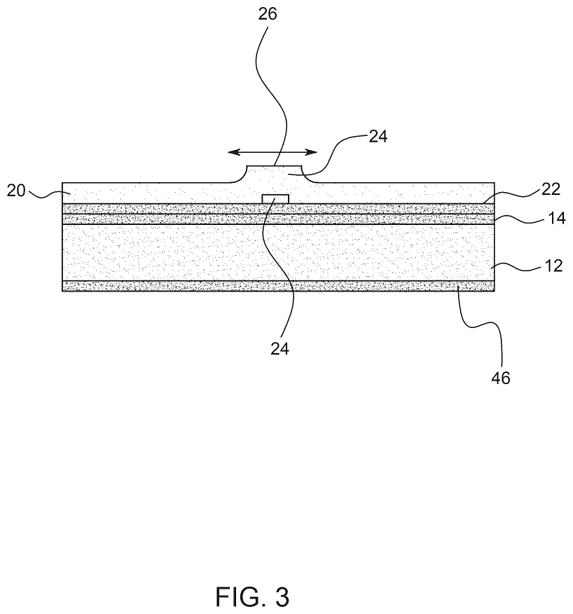

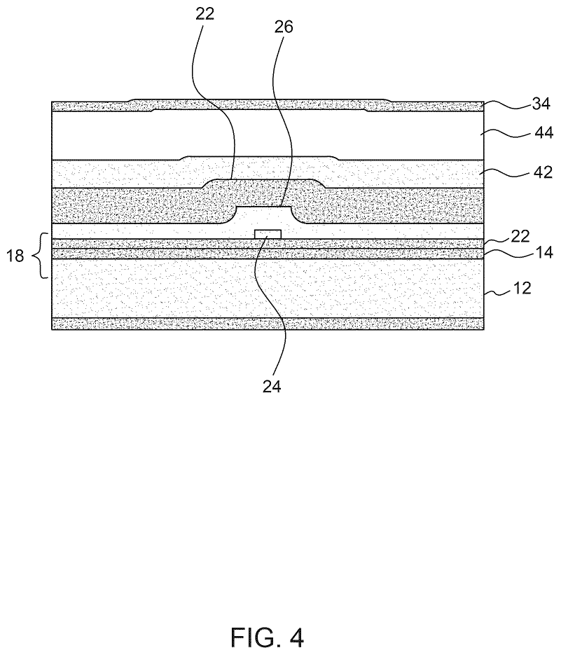

In one embodiment, illustrated in FIGS. 1A-1D, a light emitting device 10 includes:
a first mirror 12 (“DBR”), which can be a bottom mirror, and one or more active regions 14. A first active region 14 is adjacent to the first mirror 12. The one or more active regions 14 are stacked in the light emitting device 10 to provide for a selected shape structure 26 with an active region that includes a tunnel junction (TJ) 20, another active region 14, another TJ 20 and so on. As illustrated in FIG. 2 there can be a repetition of active regions 14 and TJ's 20, providing a plurality of successive active regions 14 with TJ's 20, as illustrated in FIG. 2. Each active region 14 includes quantum wells and barriers 16 surrounded by one or more p-n junctions 18, see FIG. 3. As previously recited, one or more TJ's 20 are provided. Each of an aperture 24 is provided with a selected shaped structure 26, illustrated in FIG. 4. As non-limiting examples, the selected shape structure 26 can be open or closed, geometric or non-geometric shape, and the like. The shape of the selected shape structure 26 is based on a desired optical transmission function for a desired application, as shown in FIG. 4

In one embodiment, the selected shape structure 26 is a stepped structure, see FIG. 4. The degree of the stepping can be at a variety of different angles, as determined by the etching process. The selected shape structure 26 can be substantially any geometry, including but not limited to a four corned star, circular, square and the like. As a non-limiting example, the selected shape structure 26 can be created using an inverse design. The selected shape structure 26 is selected in order to create an optimal desired far field pattern depending on the desired application. As a non-limiting example, the selected shape structure 26 is formed by etching.

In one embodiment, a first growth is used to create the bottom mirror 12, TJs 20, active regions 14, and the like. In one embodiment, a selected geometric configuration buried tunnel junction 28 is created during a regrowth process. The light emitting device 10 is created by the first growth, followed by a second or regrowth of the first growth. The first growth creates a planar structure. Additional BTJ's 28 are created from second, third and the like regrowth's. The creation of multiple BTJ's is formed during multiple regrowth processes. All of these regrowth's are independent of the first growth process.

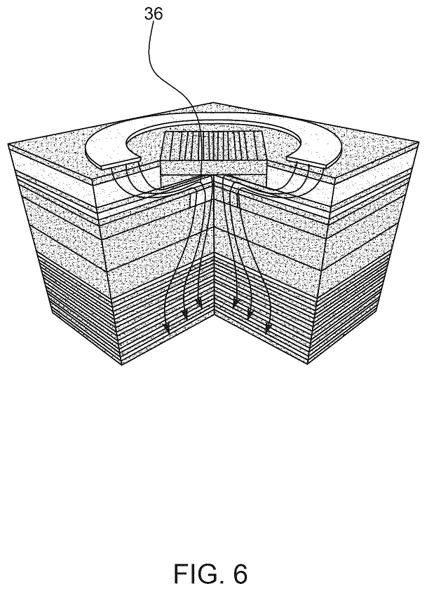

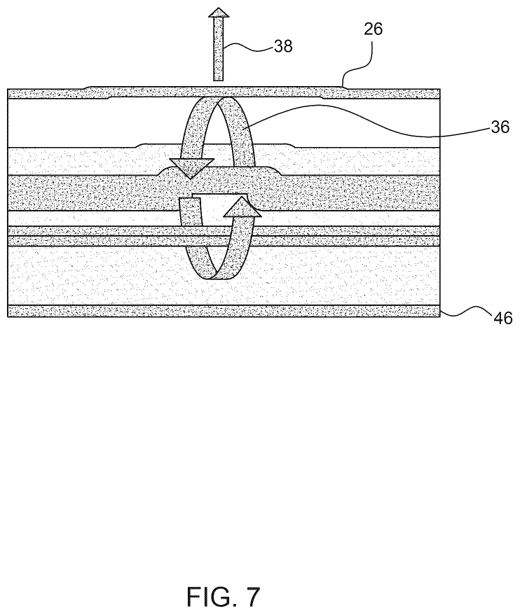

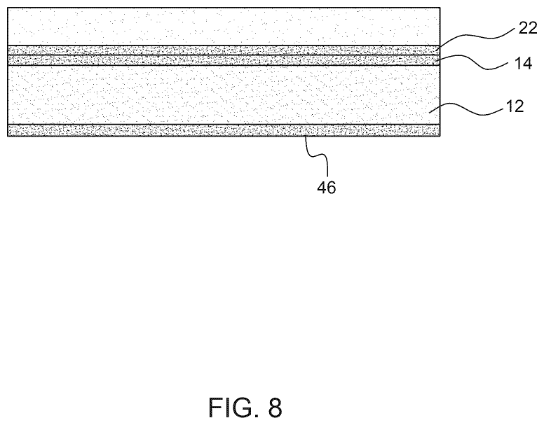

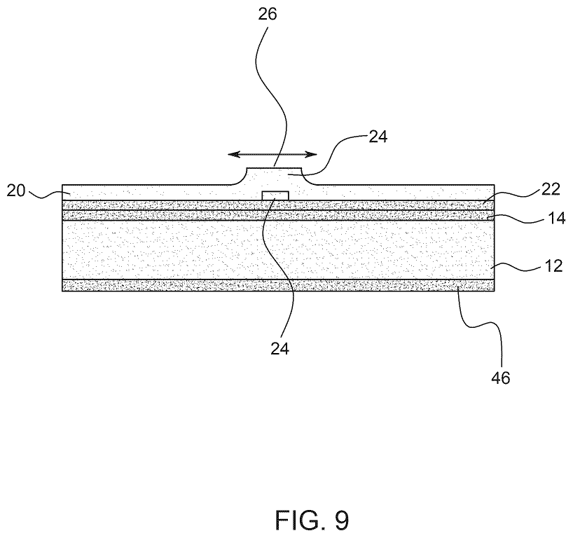

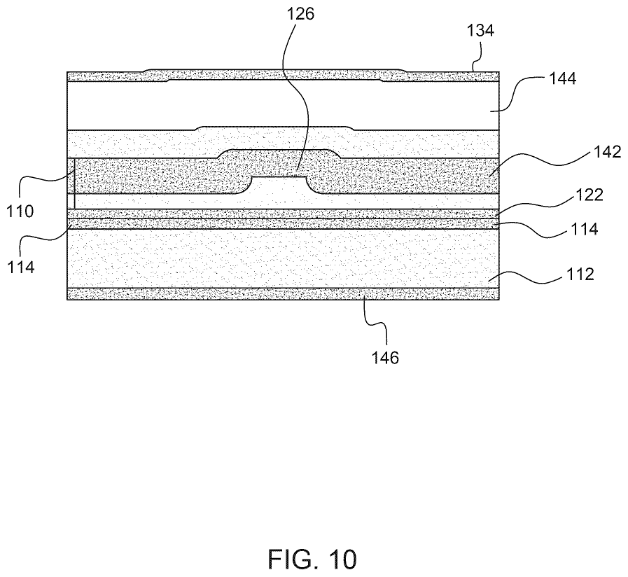

An area 30 is defined by the one or more BTJ's 28, additional TJ's 20, planar structures and/or addition BTJ's 28. A vertical resonator cavity 32 is defined by a second mirror over the electrical confinement aperture 30, see FIG. 6. A high contract grading (HCG) 34 operates as a second mirror positioned at the top of the vertical resonator cavity 32.

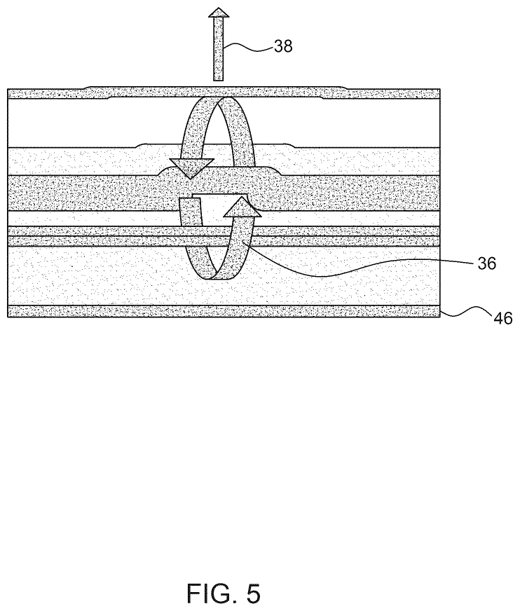

In one embodiment, the HCG 34 conformally covers the selected shape structure 26 layered on the selected shape structure 26. The HCG 34 remains anchored by a variety of different methods including not limited to: a membrane, or membrane structure, by beams after partial removal of sacrificial layer in order to move with respect to the optical resonator and modify a cavity length and consequent wavelength emitted by light emitting structure, and the like. A shape of the output beam 38, as in FIGS. 5 and 6, is determined by a geometric shape of the one or more BTJ apertures 29, and the apertures for additional TJ's 20, planar structures and/or additional BTJ's 28. and a transmission function of the HCG 34. HCG 34 is designed according to a desired optical transmission function for a desired application.

As a non-limiting example, the HCG 34 operates as a second mirror positioned over the vertical resonator cavity 32. In one embodiment, a shape of the output beam of light emitting device 10 is determined by a geometric shape of the one or more BTJ apertures 29, and apertures for additional TJ's 20, planar structures and/or additional BTJ's 28.

As a non-limiting example, the far field transmission range is application dependent. As a non-limiting example, the output beam can be a plurality of different geometries including but not limited to circular, elliptical and the like.

The selected shape structure can be created by a variety of ways. As non-limiting examples, the selected shape structure can be created can be made with a grid, a circular array of dots, a screen, hole and screens, and the like.

In one embodiment, the light emitting device 10 is a VCSEL (hereafter referred to as (“VCSEL 10”).

Relative to efficiency, the VCSEL 10 can be designed to maximize the light output relative to the desired application. Additionally, the number of BTJ's 28 created can be in response to the application.

In one embodiment, a shape of the output beam 38 is configured from a design of the electrical confinement aperture 30. As a non-limiting example, a geometry of the electrical confinement aperture 30 is slightly asymmetric to provide that the beam 36 inside the VCSEL 10 provides a Gaussian beam shape as the output beam 38 from the HCG 34.

In one embodiment, a coupling loss of the output beam 38 to a fiber or wavelength is reduced when VCSEL output beam 38 is designed to match the fiber modes. As a non-limiting example, the HCG 34 controls one or more of: an output beam 38 polarization, optical beam 3 shape and a single longitudinal wavelength. As a non-limiting example, the HCG 34 is further released from selected shape structure 26 by selective etch of spacing layers 22. In one embodiment, p-doping is on a top of the active region 14 leading to n-doping to a bottom of active region 14.

In one embodiment, at least a portion of the spacing layers 22 above the one or more BTJ apertures 29, the apertures for additional TJ's 20, planar structures and/or addition BTJ's 28, have thicknesses defined as multiples of quarter-wavelength of target lasing wavelength, in one embodiment, spacing layers and the top DBR 42 can have different thickness. As a non-limiting example, the top DBR 42 has a smaller number of layers than the bottom DBR.

Spacing layers 22 can also be used for electrical current spreading, thermal dissipation, or minimizing optical absorption, for example, but not limited to those ends.

In one embodiment, a top DBR 42 is not included in VCSEL 10. The sacrificial layer 44 of semiconductor material is removed via etching at specific locations in order to release the top mirror tuning structure. In one embodiment, the etching of the sacrificial layer 44 uses a wet or dry etch process. The sacrificial layer 44 can remain when the top mirror is not moveable as a non-tunable design and has a low index of refraction compared to a material of the top DBR 42. Successive steps are used in the growth of the VCSEL 10 and can also provide lateral optical confinement due to a graded lens effect. An epitaxial growth and a regrowth of the VCSEL 10 is achieved

In one embodiment, HCG 34 is formed of a semiconductor epitaxial layer. HCG 34 is layered on a selected shape structure 26, that can be less than 100 nm. In one embodiment, VCSEL10 produces a stable linearly polarized output defined by HCG 34. In one embodiment of the present invention, VCSEL 10 is created by a process of two independent epitaxial growths. In one embodiment, first and second epitaxial growth structures are characterized, and formed by epitaxial growth.

As a non-limiting example, the first epitaxial growth structure is on a seed substrate 46 of III-V semiconductor material and includes: the bottom mirror Distributed Bragg Reflector (DBR) 26 on a top surface of the seed substrate 46 is defined by alternate layers of high and low index of refraction. Active region, generally denoted as 24, is adjacent to first mirror 12, consisting of quantum wells and barriers 16. A plurality of layers 22 can be optional. The final layer, and the at least one TJ 20, are etched laterally to define an etched electrical confinement aperture 30 through which an electrical current flow. The HCG 34 and spacing layers 22 are formed over in a stepped format (selected shape structure 26). The etched electrical confinement aperture 30 being the one or more BTJs 28 that is a selected shape structure 26, as a result of further regrowth.

As a non-limiting example, the one or more BTJS 28 provide for VCSEL 10 current confinement in a VCSEL 10, and can be implemented for any VCSEL 10 in the SWIR band, from 650 to 1800 nm. More particularly, this can for InP based VCSELs 10 (above 1300 nm).

In one embodiment, for current confinement, the VCSEL 10 of the present invention uses the one or more BTJ's 28, additional TJ's 20, planar structures and/or addition BTJ's 2, instead of ion implant or oxide aperture.

As a non-limiting example, the VCSEL 10 with the one or more BTJ's 28, a BTJ 28 and can provide better current blocking outside of electrical confinement aperture 30 than ion implanted apertures and also provide good reliability.

In one embodiment, of the first epitaxial growth structure, the top surface of the seed substrate 46 is defined by alternate layers of high and low index of refraction.

In one embodiment, of the first epitaxial growth structure, the active region 14 is a source of light from an electrooptic effect due to a recombination of holes and electron. As a non-limiting example, the active region 14 is undoped and surrounded by one or more p-n junctions in order to promote recombination of electrons-holes.

In one embodiment, of the first epitaxial growth structure, at least a portion of the plurality of spacing layers 22 have a varied thicknesses depending on an optical design. In one embodiment, at least a portion of the plurality of spacing layers 22 are adjacent to the active region 14 or to additional spacing layers 22.

In one embodiment of the first epitaxial growth structure, the TJ 20 is a highly doped p++ layer directly on the top of a highly doped n++ layer.

In one embodiment of the first epitaxial growth structure, final layer is made of a same material of the seed substrate 46.

In one embodiment of the first epitaxial growth structure, the final layer is not included, depending on the optical design.

The TJ 20 layer is etched laterally and defines the selected shape structure 26, that is chosen based on the application. This becomes the one or more BTJs 28, a BTJ 28 additional TJ's 20, planar structures and/or addition BTJ's 28.

In one embodiment, a second epitaxial growth structure starts on a top of the first epitaxial growth structure after TJ 20 lateral etch. In one embodiment, the second epitaxial growth structure is a regrowth on top of the first stepped epitaxial growth structure, creating the one or more BTJs 28 from the one or more TJs 20, This becomes the one or more BTJs 28, additional TJ's 20, planar structures and/or addition BTJ's 28.

In one embodiment, the second epitaxial growth structure includes: spacing layers 22; an optional top DBR 42 mirror; a sacrificial layer; a top layer, which supports the top mirror manufacturing; and extra layers for supporting metal contacts.

In one embodiment, the p-doping is on a top of the active region 14 leading to n-doping to the bottom of the active region 14. In one embodiment, this is reversed and the n-doping is on the top of the active region 14, the p-doping is on the bottom.

In one embodiment of the second epitaxial growth structure, the second epitaxial growth structure starts on a top of the previously processed semiconductor stack.

In one embodiment of the second epitaxial growth structure, the second epitaxial growth structure includes spacing layers 22. As a non-limiting example, these spacing layers 22 have a varied thickness depending on the optical design.

In one embodiment of the second epitaxial growth structure, the spacing layers 22 are above the etched TJ 20 aperture. In one embodiment, the etch of the TJ 20 can be etching into the layers below the TJ 20. The TJ 20 and the layers below it can be fully etched, partially etched and the like. As a non-limiting example, the profile of the etch can be based of the selected application, and sidewalls can be created with sidewall angles of 20 to 90 degrees. the sidewalls of the etch can be created based on etching recipe, RF power, the type of chemicals used, crystal dependence, as well the desired end application. In one embodiment of the second epitaxial growth structure, they are added to complete a multiple of quarter-wavelength of a target lasing wavelength. In one embodiment, they have different thickness. In one embodiment, the spacing layers 22 are also used for electrical current spreading, thermal dissipation, or minimizing an optical absorption, and the like.

In one embodiment of the second epitaxial growth structure, the second epitaxial growth structure has a possible top DBR 42 mirror on top of a seed substrate 46, defined by alternating layers of high and low index of refraction of semiconductor. This top DBR 42 is not mandatory and, as a non-limiting example, has a much smaller number of layers when compared to bottom DBR 26, if it is included in the stack, and optimized based on the application, as well as two beam parameters. In one embodiment, the two beam parameters are reflectivity and the wavelength bandwidth of the mirror.

In one embodiment of the second epitaxial growth structure, the second epitaxial growth structure can be application specific, and include specific spacing sacrificial layer of semiconductor material under top mirror. This can be removed via etching at specific locations in order to release the top mirror tuning structure. In various embodiments, the etching of the sacrificial layer can be achieved with a wet or dry etch process. In one embodiment, this layer can alternatively remain in the structure if the top mirror is not supposed to move (non-tunable design) and has a low index of refraction compared to the top mirror material. In one embodiment, the index of refraction is application and can be as large as possible.

In one embodiment of the second epitaxial growth structure, the top mirror layer is processed during a post regrowth using an etching process and becomes a periodic structure designed to resonate and work as a mirror at specific wavelengths.

In one embodiment, the periodic structure is a high contrast metastructure (HCM) or high contrast grading HCG 34, known as subwavelength grading, or a photonic crystal structure. In one embodiment, the mirror effect requires a lower or a same index of refraction around the periodic structure, which can be air, if released, or other semiconductor or dielectric material, if not released. When it is a tunable structure, the periodic structure is actuated in order to change the optical length of optical cavity vian external input that causes movement of the periodic structure. In one embodiment, an external input is based on thermal, electrostatic or piezoelectric excitation. In one embodiment of the tunable design, tunability can also be achieved by heating the VCSEL 10 or changing its driving current.

In one embodiment of the second epitaxial growth structure, the second epitaxial growth structure extra layers are provided that are designed for supporting metal contacts.

In various embodiment, VCSEL 10 is at least one of: an air-cavity-dominant (ACD) or a semiconductor-cavity-dominant (SCD) design. In one embodiment, the regrowth process is configured to be nearly conformal and increase the lateral dimension of initial BTJ step as defined by the electrical confinement aperture 30 during deposition of epitaxial layers in the second epitaxial regrowth, in one embodiment, the regrowth process decreases a height of this step as more and more layers are added. In one embodiment, at the end, the periodic structure for the first mirror 12 is defined on a selected shape structure 26, which, however, does not have to be necessarily a plane.

As non-limiting examples, the successive steps can provide for lateral optical confinement due to a graded lens effect and improve overall efficiency of VCSEL 10.

In one embodiment, the epitaxial growth and regrowth of the VCSEL 10 can be achieved by conventional III-V epitaxy, such as molecular beam epitaxy (MBE) or metallo-organic chemical vapor deposition (MOCVD) or other techniques including but not limited to LPE, SPE and the like. While MBE can provide better control of each atomic layer, MOCVD can provide a better step coverage.

The HCG 34 can provide strong polarization control since the grading, HCG 34, acts as a polarizer, i.e., only stable linear polarization is transmitted through the grading, HCG 34, In one embodiment, the non-planar structure is formed due to the regrowth process which is started on selected shape structure 26 with TJ 20 delimited by a top of a selected geometric structure, including but not limited to a mesa.

In one embodiment, the one or more BTJ apertures 29 that are created during one or more regrowth. After regrowth, etched TJ 29 becomes BTJ 20, a buried TJ.

In one embodiment, the HCG 34 is configured to reflect a first portion of light 36 back into the vertical resonator cavity 32. A second portion of light 38 is transmitted as an output beam from the light emitting structure 10. As a non-limiting example, every time the first portion of light 36 is reflected into the vertical resonator cavity 32, portions of light, constituting the second portion of light 38, come out of the light emitting structure 10. As a non-limiting example, the first portion of light 36 bounces around the vertical resonator cavity 36, with a portion coming out as the second portion of light 38.

As a non-limiting example, after sufficient thick regrowth, selected shape structure 26 can become planar, a plane plateau, and the like. It can easier to control processing of the periodic structure. In one embodiment, HCG 34 can be manufactured on a curved of stepwise layer, as far as continuous. In one embodiment, a final dimension of the plane plateau is originated from the epitaxial growth on top of electrical confinement aperture 30 and depends on a thickness of intermediary layers and growth conditions. As a non-limiting example, this thickness can be in the range of 10 um to 20 um for a starting electrical confinement aperture 30 with a 3-um diameter.

In one embodiment, VCSEL 10 is integrated in line detector behind it for FMCW applications, where the reflected light reflects into the VCSEL, causing optical feedback. The detector behind the VCSEL detects power oscillations due to the feedback and the signal can be used to reconstruct the 3D environment.

In one embodiment, a photodetector is at a side of an air gap in s tuning cavity as a detector. As a non-limiting example, the semiconductor, to the side of the air gap in the tunable structure, act as the photodetector and collect scattered light from the HCG and VCSEL body interface due to non-idealities. This offers some advantage to the application in 1) because the photodetector is outside of the cavity and does not contribute any additional feedback, simplifying the signal processing.

In one embodiment, a sensing apparatus includes a VCSEL 10 including one or more active regions 14. Each active region 14 has quantum wells and barriers 16. Active regions 14 are surrounded by one or more p-n junctions, the one or more active regions including a selected shape structure each with a tunnel junction (TJ). One or more apertures are provided with the selected shape structure. One or more buried tunnel junctions (BTJ) 28 or oxide confine aperture, additional TJ's 20, planar structures and/or additional BTJ's 28 are created during a regrowth process that is independent of a first growth process. An output of VCSEL 19 is determined in response to an application of the light emitting device. In one embodiment, bottom DBR 12 can include an HCG and dielectric coating with one or more layers of the dielectric coating.

In one embodiment, the dielectric coating is a stack of high/low index layers As non-limiting examples, the dielectric coating can be a stack of 5-10, 6-10, 7-10, 10 high/low index pairs of dielectric. The dielectric provides a wider tuning range. This provides a broad wavelength mirror. In one embodiment, the top DBR mirror 42 is s a moveable mirror.

With tunable, a specific wavelength is targeted and VCSEL 10 remains at that wavelength. Changing cavity 32 size allows VCSEL 10 to move, and physically change the cavity 32 size for each application In one embodiment, wavelength locking control is provided. This requires an external reference including but not limited to a chip that taps the light. This is then coupled to a reference system. The reference system and the chip are external to the VCSEL In one embodiment, atomic plots are used for the reference system.

As non-limiting examples, ranges of output power can be: one additional active region (or junction) can add ~1× power, and the laser could become 2× more powerful. However, as it also adds extra heat coming from additional optical power inside cavity, efficiency goes down. Thus, by adding more junctions we can have more power but to the price that the laser cannot work continuously anymore. Typical multi-junction VCSELs work on pulsed mode and it is very hard to predict range of output power. As a rule of thumb, we can say that 2 junctions will have 2× more power on pulsed operation, 3 junctions 3×. As one increases the number of junctions, pulses have to become faster and intervals longer.

In one embodiment, the output power is from 1 milliwatt to 5 milliwatts per aperture. In one embodiment, multi junction devices have output powers of 10-20 milliwatts, 12-17 milliwatts, and 15 milliwatts. In one embodiment, the oxide confinement defines aperture 24 for electrical current confinement.

As a non-limiting example, a number of quantum wells can be the same in all junctions, but not necessarily. Individual quantum well design can also be slightly different in order to broaden spectral material gain and increase tuning range.

In one embodiment, a regular VCSEL apparatus 10 includes current injection via the one or more BTJs 28, TJ's 20, planar structures and/or additional BTJ's without ion implantation.

As a non-limiting example, VCSEL apparatus 10 includes one or more BTJ's 28 and is tunable. In one embodiment, formation of the one or more BTJ's28 TJ's 20, planar structures and/or additional BTJ's, requires an etch of a TJ aperture and regrowth of a top semiconductor epitaxial structure. As a non-limiting example, the process results on a non-flat surface, not suitable for an HCG 34. Moreover, uneven surface and regrowth process causes additional strain which also prevents MEMS manufacturing. In one embodiment, this requires relaxation of epitaxial growth through careful design of regrowth and MEMS actuation on the non-planar surface.

In one embodiment, VCSEL 10 formation includes the steps of:
(i) growing a semiconductor active cavity material consisting of a multi-quantum well layer stack sandwiched between bottom and top spacer regions, the top spacer region terminating with a p-layer and a p++/n++ tunnel junction grown on top of the p-layer, each of the p++- and p-layer presenting a p-type layer;
(ii) etching the active cavity material formed in step (i) to form a top of a selected geometric structure such as a mesa, including at least the upper n++ layer of the tunnel junction emerging from the underlying p-type layer, thereby creating a structured surface of the active cavity material formed by the upper surface of the upper surface of the p-type layer outside a top of a selected structure such as a mesa;
(iii) applying a wafer fusion between the structured surface of the active cavity material and a substantially planar surface of a n-type semiconductor layer of a first distributed Bragg reflector (DBR) stack, thereby causing deformation of the fused surface around a top of a selected structure such as a mesa, and defining an electrical confinement aperture 30 for electrical current flow therethrough, the electrical confinement aperture 30 including a top of a selected structure such as a mesa, surrounded by an air gap between the deformed fused surfaces and defining an active region 14 of the device;
(iv) forming a second DBR stack on the surface of the active cavity material opposite to the structured surface;
(v) forming ohmic contacts on the VCSEL device structure to enable the electrical current flow through the electrical confinement aperture 30 to the active region 14.

In one embodiment, VCSEL apparatus 10 is made with the following processes: epitaxial growth of VCSEL up to tunnel junction (TJ) layer 20; development of TJ etches; identify etchants compatible to the TJ design 20; and development of regrowth process.

Surface compatibility to regrowth-not all surfaces or all materials are suitable for seeding the regrowth process-there is a trade off in between the best design for the TJ 20 and a design good enough which is suitable for regrowth.

Regrowth has to be carried long enough in order to planarize the surface where HCG 34 is manufactured on one or more of: VCSEL regular processing; growth of bottom VCSEL and then: etch of TJ 20, leaving some p++-target is 5-10 nm and regrowth of top VCSEL from etched seed layer.

Then there is: a regrowth of n-InP with grading; a regrowth of top DBR 42 (if included in design), sacrificial layer and HCG 34; followed by standard VCSEL manufacturing.

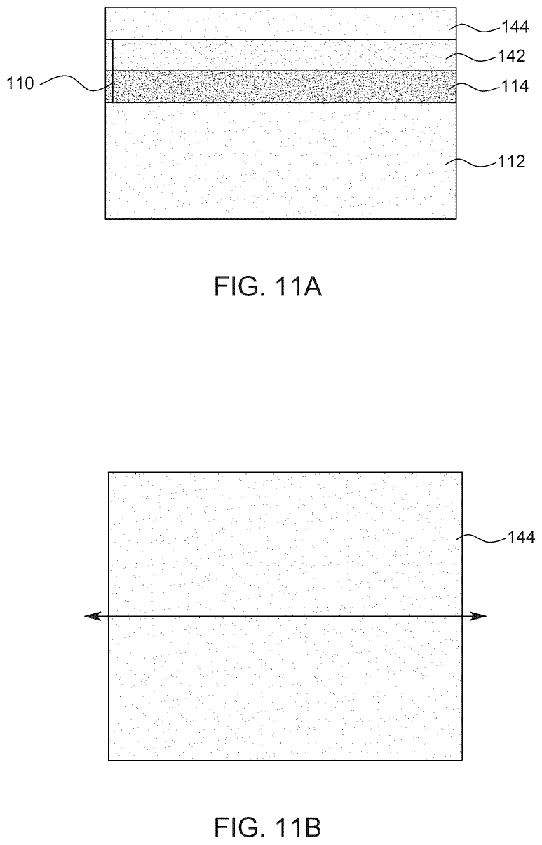

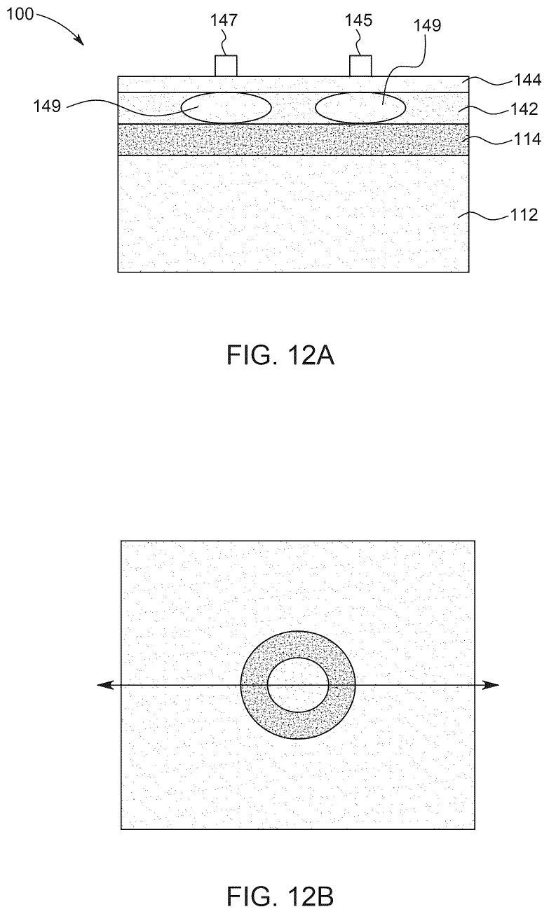

In one embodiment a regular VCSEL 10 epitaxial structure includes an etched post between an active region 112 and a sacrificial layer 114. FIGS. 11A and 11B illustrate a first regrowth including a bottom DBR 116, active layer (region) 112, top DBR 118/other spacing layers 120. The sacrificial layer 114 has a thickness now greater than 100 nm. The optical confinement is defined by the etched post 110 which acts as a waveguide. FIGS. 12A and 12B illustrates one embodiment of implantation.

In one embodiment, an etched post 110 and regrowth provide lateral current and optical confinement, small volume and increased efficiency for more demanding applications, such as very high-speed modulation and coherent communication. The increased efficiency is achieved because the optical wave and the lateral currents overlap.

Instead of etching the post 110 for confinement in the optical path via mesa etch and regrowth, the optical path is preserved and modify its boundaries for optical confinement. The existence of the sacrificial layer 114 in the present invention favors this new approach as the final interface of regrown material in the optical path is etched away. This preserves optical quality. As a non-limiting example, the manufacturing requires integration and compatibility of several different processes, not required for the conventional semiconductor as-grown DBR.

Regrowth of the sacrificial layer 114 around a small mesa 122 step brings much smaller complication when compared to previous approaches of buried heterostructures with steep walls more than 2× taller (3-4 um) than mesa 122 (0.8 um). In one embodiment, a full VCSEL is grown up to a thin SAC layer (100nm of Al0.22Ga0.25In0.53As with 100 nm InP cap). As illustrated in FIGS. 12A and 12B implantation is then proceeded for current confinement under the metal contact pads. In illustrates the etched post 110.

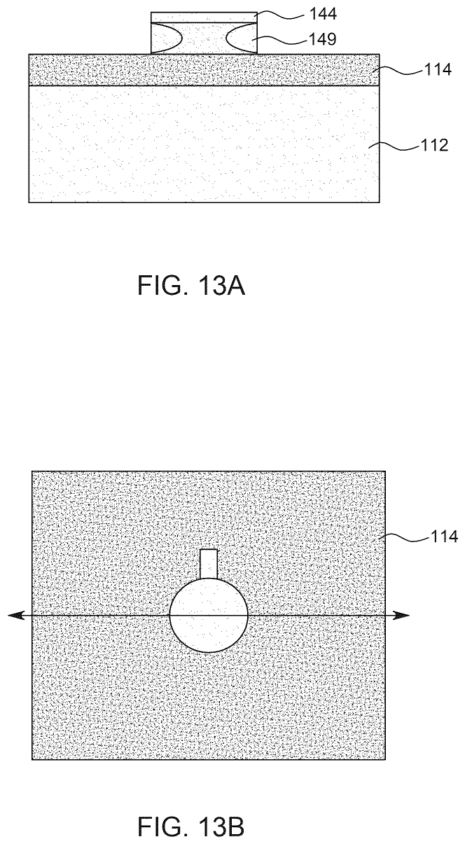

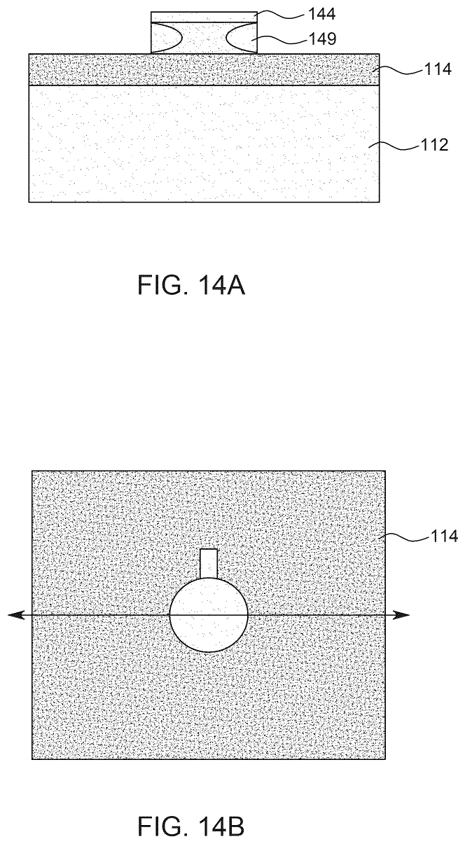

In one embodiment, implantation is done on a structure with the bottom DBR 116, active layer 112 and a thin sacrificial layer 114. The implantation is done on a layer between the active layer 112, and the sacrificial layer 114. An implant mask is used. The structure, including the bottom DBR 116, active layer 112, sacrificial layer 114 and mesa 122 position therebetween, is then dry etch down a very tiny mesa 122 for high-speed (radius of 5-10 um), up to InP layers, close to a TJ interface FIGS. 13A and 13B. The mesa 122 having an increasing radius up to a top layer. Optical monitoring can be used. Referring to FIGS. 14A and 14B, atomic layer deposition (ALD) is performed only on side walls of etched post 110. This protects the etched post 110 from further release, increased reliability and isolation from surrounding air.

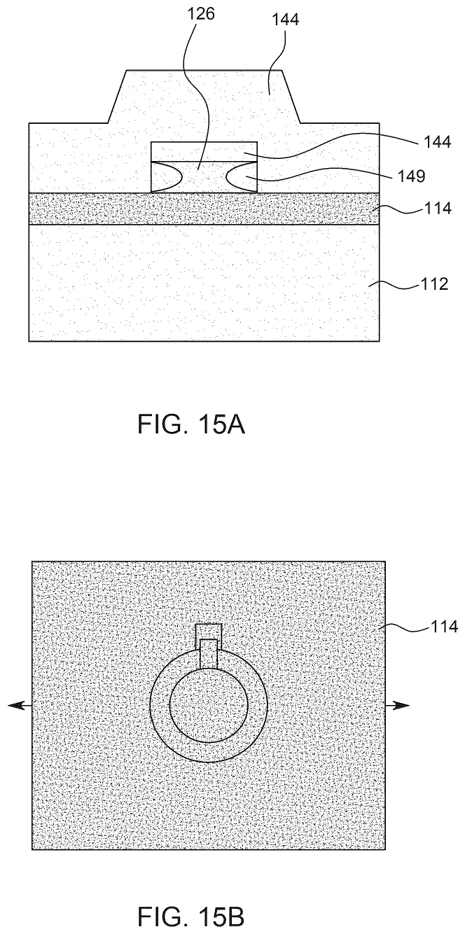

In one embodiment, illustrated in FIGS. 15A and 15B, ALD wall protection is used for further selective etch on release, and for reliability.

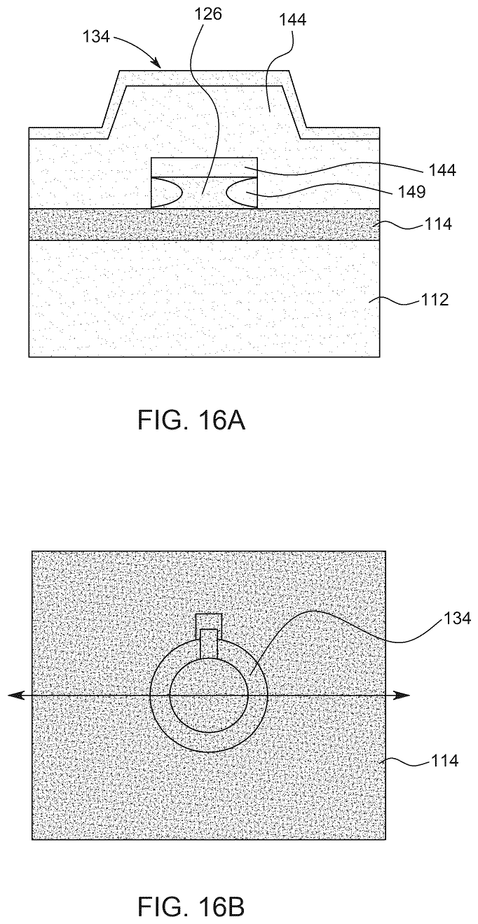

A full regrowth of a full sac layer +HCG on top is performed, as illustrated in FIGS. 16A and 16B, with the interface etched away. InP regrowth is a regrowth around the etched post 110. This is later removed, along with the sacrificial layer 114.

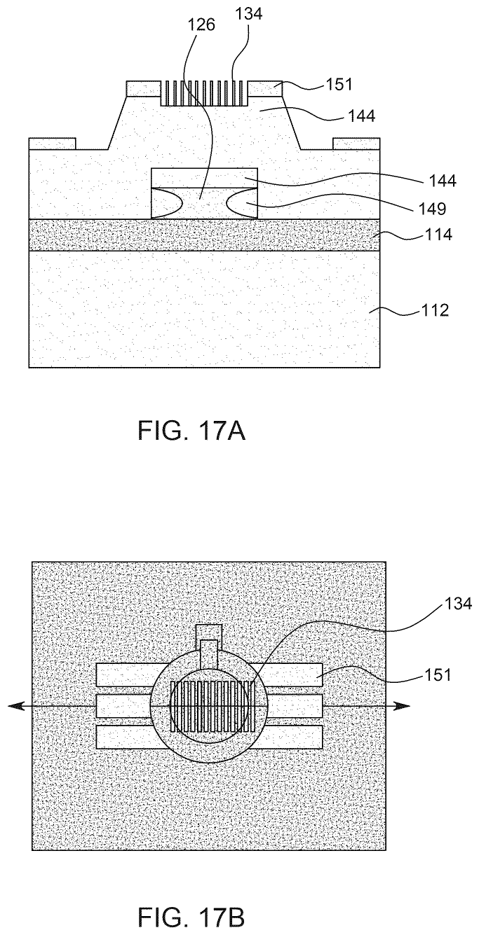

The regrowth interface is out of the optical/current paths. In one embodiment, the regrown layer, the new growing layers cause some defects on the crystal before arriving at the HCG layer. The structure includes the bottom DBR 116, active region (layer) 112, a thin sacrificial layer 114, a mesa 122 therebetween, and a regrowth sacrificial layer 114. In one embodiment, the mesa 122 grows laterally during regrowth for a slightly bigger than mesa 122 HCG, FIGS. 17A and 17B As a non-limiting example, a vertical step on the MEMS beams is not a problem. There is no need for implantation confinement.

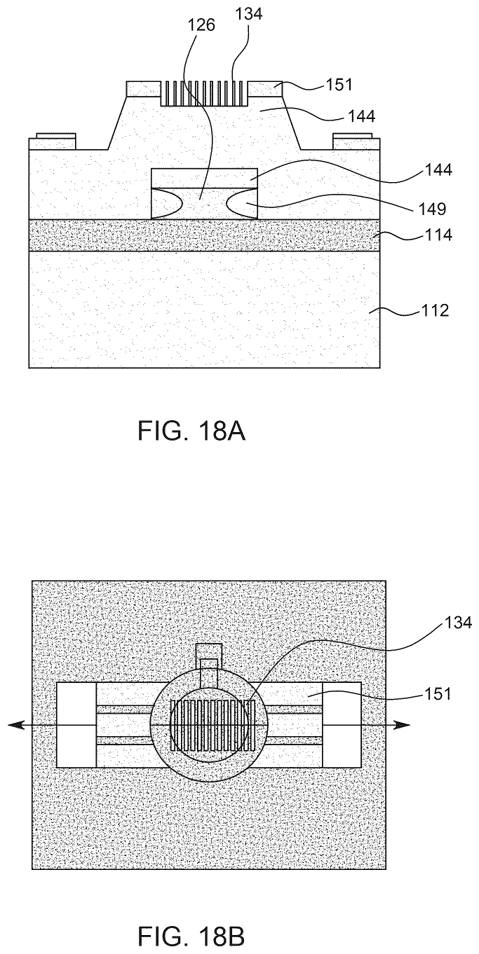

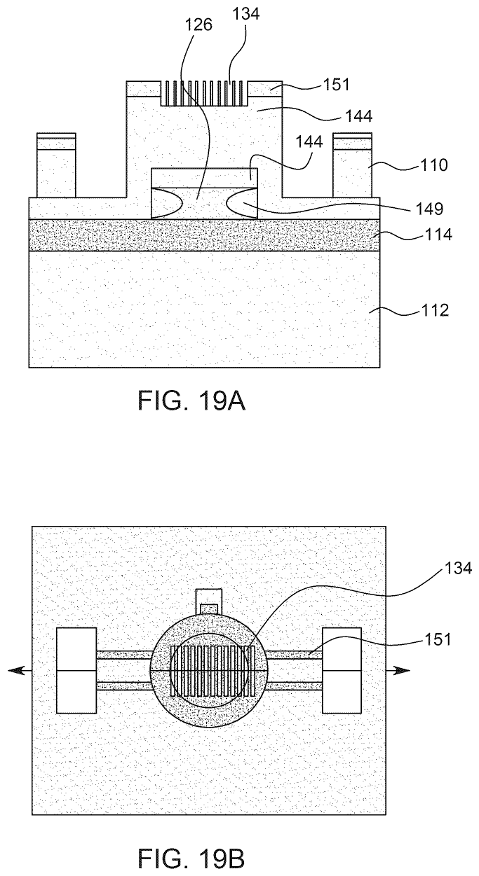

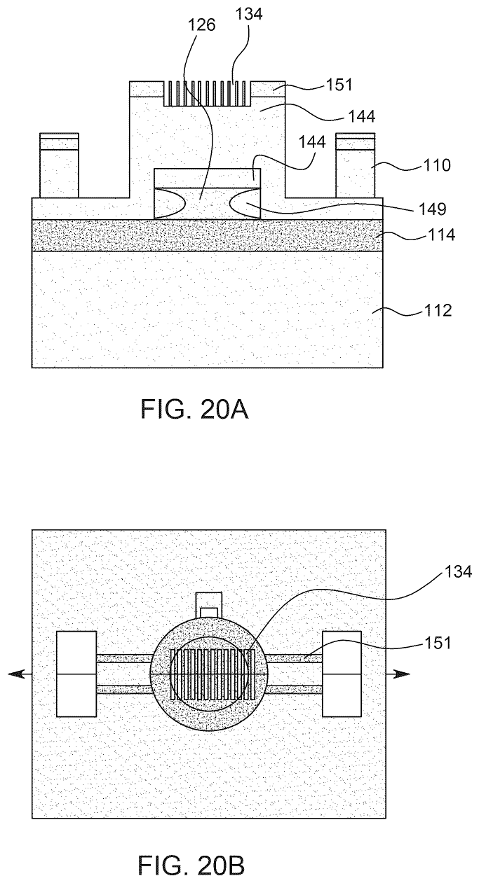

In one embodiment, mesa 122 confinement is provided, e. g, implantation is required only for contact isolation and current injection from the top of the mesa 122. In one embodiment, normal processing is completed for adding contact pads, which as a non-limiting example, the contact pads can be metal 3 and polyimide. MEMS anchors are on either side of the mesa 122, FIG. 18A an 18B. In one embodiment, a second mesa etch is provides, FIGS. 19A and 19B, following by an MI cut, FIGS. 20A and 20B.

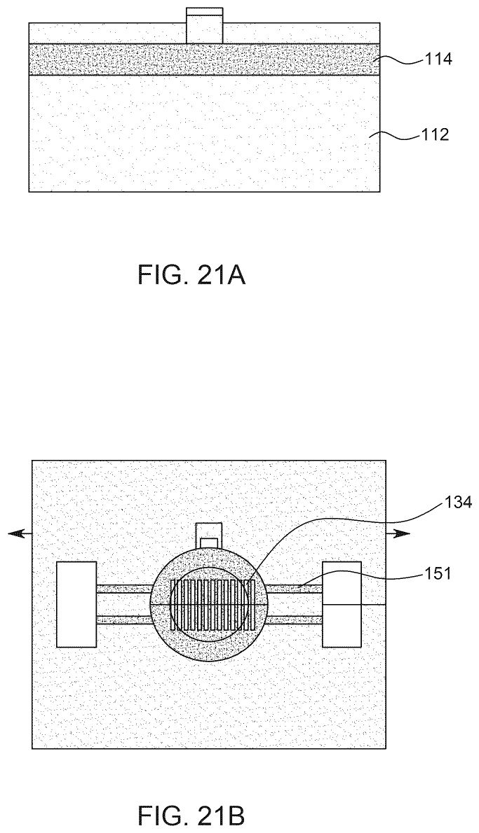

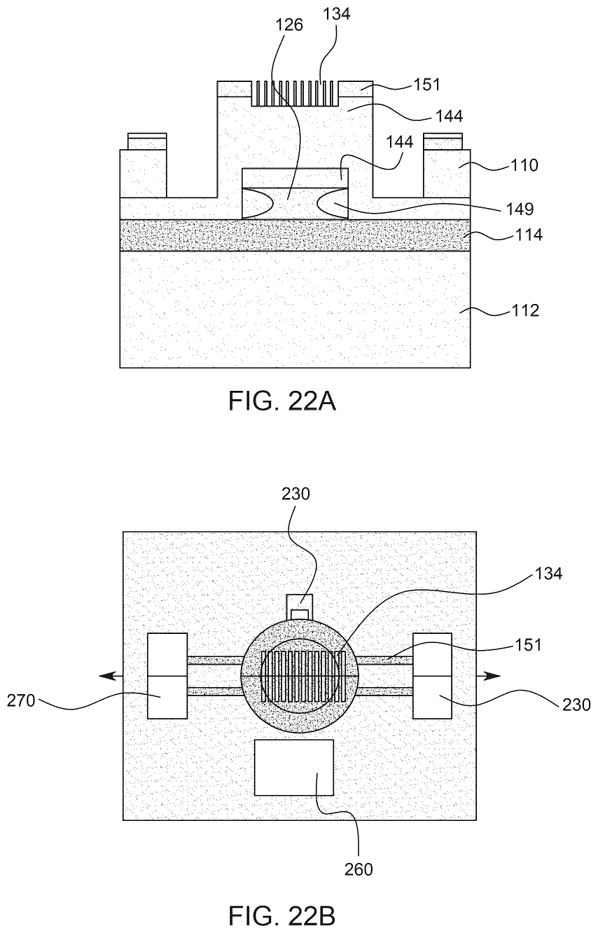

An opened access, M2, to the bottom DBR 116 contact is created, FIGS. 21A and 21B. This is followed by a release, FIGS. 22A and 22B. The release can be at the MEMS beams.

In one embodiment, the 38 of the VCSEL laser has a long wavelength, including but not limited to from . 720 micron to 1.7 microns, and in one instance, 1.365 microns.

In one embodiment, VCSEL 10 products an output beam with a wavelength of 1550 nm is chosen due to its high signal-to-noise ratio (SNR) for glucose signals.

In one embodiment, the output 38 of the VCSEL laser is a long wavelength, at least partially created from indium phosphide structure or material in the VCSEL laser 10. As a non-limiting example, VCSEL laser 10 can include an indium phosphide substrate 46, or a substrate 46 that at least partially includes indium phosphide.

In one embodiment, VCSEL laser 10 is coupled to or includes a top DBR 42 or high contrast grading (HCG) 34. As a non-limiting example, bottom DBR 12 is a semiconductor DBR or a combination of a semiconductor DBR with a dielectric coating. In this manner, VCSEL 10 can include a dielectric coating. As a non-limiting example, the dielectric coating improves a broadening of a tuning range of the VCSEL laser 10.

In one embodiment, VCSEL laser 10 operates in a single mode or a multi-mode operation. As a non-limiting example, dimensions of the aperture and HCG 34 are contributing factors to the single mode operation.

In one embodiment, VCSEL laser 10 can deploy multiple tunnel junctions to enhance the output 38 of VCSEL laser 10.

As a non-limiting example, buried tunnel junctions (BTJ) improve an energy efficiency of VCSEL laser 10.

As a non-limiting example, a wavelength of the VCSEL laser output 38 can be swept to provide improved resolution. In one embodiment, the VCSEL laser output 38 is swept by modulating an HCG grading 34 up and down, e. g, its up and down movement relative to a top of VCSEL laser 10, wherein when the HCG 34 moves closes to non-extended portion relative to a top of VCSEL laser 10, a wavelength of the output 38 changes and returns closer to an original output 38 of the VCSEL laser, without extension of the grading.

In one embodiment, a mem's structure is coupled to the HCG grading 34 or top DBR 42 to create a swept source. As a non-limiting example, modulating a VCSEL laser output 38, in combination with a sweeping of wavelengths of the VCSEL laser output 38 allows for higher resolution and reduces at least a portion of atmospheric interference of VCSEL laser operation.

In one embodiment, multiple tunnel junctions increase an optical power of the VCSEL laser 19. These junctions are provided in a body of the VCSEL laser 10.

In one embodiment, a semiconductor is included with a modulator to allow a swept source to be modulated. As a non-limiting example, the modulation of the VCSEL laser turning the VCSEL laser on and off. As a non-limiting example, the modulation is from about 1 to 50 G. In another embodiment, the modulation is greater than 50 G.

As a non-limiting example, an optical photonic integrated circuit (PIC) is coupled to the VCSEL laser. The VCSEL laser can be mounted on the PIC. In one embodiment, a plurality of VCSEL lasers are mounted on the PIC.

In one embodiment, HCG 34 acts as a partial mirror. As a non-limiting example, HCG 34 can operate as a second mirror HCG 34 is positioned at the top of the vertical resonator cavity 32, following removal of at least a portion of the sacrificial layer 44. HCG 34 is positioned at the top of the vertical resonator cavity 32. As a non-limiting example, HCG 34 is positioned on a top of the mesa. in one embodiment, a plurality of support elements, anchors, hold HCG 34 in place. HCG 34 then moves, typically up and down relative to cavity 32.

Two or more support elements are provided. Support elements provide support for HCG 34. As a non-limiting example, support elements can have dimensions of 50 microns long, one micron wide. Their thickness is determined by a growth of HCG 34 layer. Superficial layer 44 is defined by epitaxial growth. As a non-limiting example, HCG deformation is provided under different points of operation. For broad tuning, HCG 34 deforms and moves away from cavity 32. A period and duty cycle of HCG 34 are determined by the amount of deformation. As a non-limiting example, a thickness of support elements define their function. A modification of support elements occurs when there is an actuation. This causes support elements to bend and HCG 34 deforms. In one embodiment, the support elements 45 are not straight beams. In various embodiments, there can be variations of support elements to modify HCG 34 deformation.

A good portion of sacrificial layer 44 is substantially removed, at least a portion creating anchors for HCG 34.

As a non-limiting example, a main actuation of HCG 34 can include but not be limited to: electrooptic, with an application of voltage across the surface of HCG 34, thermal, piezoelectric and the like. an air gap is between the top surface of vcsel laser 10 and HCG 34. Top DBR is not necessary.

When a voltage is applied, anchors do not allow current to flow over and cause HCG 34 to move. This creates charging between HCG 34 and the mesa 122. When voltage is increased, an attraction vis electro static actuation can be increased. As a non-limiting example, actuation is achieved with piezoelectric, thermal and the like. This provides tuning that depends of the wavelength and power. In one embodiment, tuning up to 100 nm is provided.

In one embodiment, electrostatic actuation is used by application of a voltage

As a non-limiting example, less than 1% of the output beam is emitted from VCSEL laser 10.

The remaining portions of sacrificial layer 44 not removed provide anchoring points for HCG 34. As a non-limiting example, portions of the sacrificial layer 44 remain to provide anchoring in order for HCG 34 to move.

There can be two or more support elements coupled to a remaining portion of sacrificial layer 44. In one embodiment, four support elements are provided. Different directions of moving HCG 34 are provided. In various embodiments, HCG 34 can have a variety of different geometries, including but not limited to hexagonal, octagonal, a double frame design and the like.

In one embodiment, the output 38 of the VCSEL laser has a long wavelength, including but not limited to from 1 micron to 1.7 microns, and in one instance, 1.365 microns.

In one embodiment, the output 38 of the VCSEL laser is a long wavelength, at least partially created from indium phosphide structure or material in the VCSEL laser 10. As a non-limiting example, VCSEL laser 10 can include an indium phosphide substrate, or a substrate that at least partially includes indium phosphide.

In one embodiment, VCSEL laser 10 is coupled to or includes a top DBR 42 or high contrast grading (HCG) 34. As a non-limiting example, bottom DBR 12 is a semiconductor DBR or a combination of a semiconductor DBR with a dielectric coating. In this manner, VCSEL 10 can include a dielectric coating. As a non-limiting example, the dielectric coating improves a broadening of a tuning range of the VCSEL laser 10.

In one embodiment, VCSEL laser 10 operates in a single mode or a multi-mode operation. As a non-limiting example, dimensions of the aperture and HCG 34 are contributing factors to the single mode operation.

In one embodiment, VCSEL laser 10 can deploy multiple tunnel junctions to enhance the output 38 of VCSEL laser 10.

As a non-limiting example, buried tunnel junctions (BTJ) improve an energy efficiency of VCSEL laser 10.

As a non-limiting example, a wavelength of the VCSEL laser output 38 can be swept to provide improved resolution. In one embodiment, the VCSEL laser output 38 is swept by modulating an HCG grading 34 up and down, e. g, its up and down movement relative to a top of VCSEL laser 10, wherein when the HCG 34 moves closes to non-extended portion relative to a top of VCSEL laser 10, a wavelength of the output 38 changes and returns closer to an original output 38 of the VCSEL laser, without extension of the grading.

In one embodiment, a mem's structure is coupled to the HCG grading 34 or top DBR 42 to create a swept source. As a non-limiting example, modulating a VCSEL laser output 38, in combination with a sweeping of wavelengths of the VCSEL laser output 38 allows for higher resolution and reduces at least a portion of atmospheric interference of VCSEL laser operation.

In one embodiment, multiple tunnel junctions increase an optical power of VCSE 10. These junctions are provided in a body of the VCSEL laser 10.

In one embodiment, a semiconductor optical amplifier is included with a modulator to allow a swept source to be modulated. As a non-limiting example, the modulation of the VCSEL laser turning the VCSEL laser on and off. As a non-limiting example, the modulation is from about 1 to 50 G. In another embodiment, the modulation is greater than 50 G.

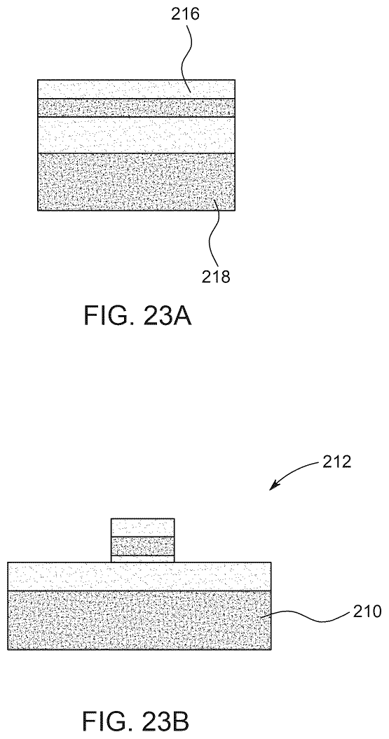

In one embodiment, illustrated in FIGS. 23A and 23B, semiconductor optical amplifier with optical fiber 200 (SOA) includes tunable VCSEL 10, a cooler 210, first heatsink 212, ASIC or driver controller 214, a thermistor 216, one or more lenses 218, one or more optical isolators 219, one positioned at a front of tunable VCSEL 10 and the second at a front of SOA 210., beam splitters 222, monitor 224, balance detectors 226, photodiode monitors 227, optical bench 228, a second thermal electric cooler 230, and a second heatsink 231. As a non-limiting example, the semiconductor optical amplifier 200 is configured to be coupled to an optical fiber 229. Also provided is a K-clock 231.

First cooler 210 provides precise control of temperature for the tunable VCSEL 10 for precision wavelength control. As a non-limiting example, cooler 210 can be a thermal electric cooler 210. Cooler 210 provides cooling to all or a portion of SOA 200. First heatsink 212 removes heat from cooler 210. Driver controller 214 provides control for a plurality of different parameters of SOA 200 including but not limited to: sweep control of tunable VCSEL 10 for wavelength use and selection, controlling temperature of SOA 200 and tunable VCSEL 10, feedback of the K-clock 231, thermistor 216 and the like to measure temperature. The one or more lenses 218 focus output beam 38. Optical isolator 220 receives polarized output beam 38 rotates it, and reduces and/or prevents all or a portion of polarized output beam reflecting back into tunable VCSEL 10 and SOA 210. Beam splitter 222 splits output beam 38. Monitors 224, which can be a photo-diode monitor, monitors power of output beam 38. As a non-limiting example balance detector 226 and optical bench 228 provide support and stability for SOA 200. Second cooler 230 cools SOA 200. SOA 200 is cooled while maintaining high temperature control of VCSEL 10. Cooler 200 provides temperature control of tunable VCSEL 10. Cooler 230 provides temperature control of SOA 210. As non-limiting examples, temperature are controlled for a variation in temperature of plus or minus 0.1 degrees for tunable VCSEL 10, and 1 degree variation for SOA 210. As a non-limiting example, the semiconductor optical amplifier 200 is configured to be coupled to optical fiber 229.

ASCIC 214 controls the temperature of coolers 200 and 230. This provides swept source for drive to tunable VCSEL 10, and supplies the drive to SOA 210 as well as a feedback loop from K-clock 231. The feedback loop provides for linearization It allows sweep rates to be changed and optimizes power out into SOA 230, as well as flattening SOA power out-put

As a non-limiting example, K-clock 231 provides a sweeping of tunable VCSEL 10 as a non-linear function, a square root. The K-clock 231 provides a feedback loop of a sweeping of tunable VCSEL sweeping, to provide linearly of tunable VCSEL 10 sweep.

Swept-source optical coherence tomography (SS-OCT) enables depth-resolved interferometric imaging based on frequency-swept light sources. It has a number of attractive features compared to the time-domain OCT (TD-OCT) or spectral-domain OCT (SD-OCT) technologies. It can provide a higher imaging speed in keeping fine resolutions and good sensitivity. It can easily work with a balanced photodetector which greatly reduces annoying imaging artifacts coming from common-mode noise components. The long-wavelength bands that low-cost silicon image sensors do not support in SD-OCT can be better utilized by SS-OCT for deeper penetration depths in tissue. SS-OCT is also superior in getting long axial ranges of imaging above centimeters [6,7]. Because of those features, SS-OCT has become one of the most promising optical modalities for medical imaging applications.

Continuously or nearly continuously swept source changes its optical frequency of the laser in a sweep. The sweep characteristics are evaluated by a number of factors including the speed, range, implementation cost, sweep linearity and repeatability. In most cases, swept source produces an undesirable nonlinear relation of time to optical frequency and may need OCT signal reassignments for best resolution performance. At the same time, the time-to-frequency relation may exhibit sweep-to-sweep variations which result in uncertainty of frequencies for acquired signals of spectral interferogram. The properties of the inter-sweep variations are subject to the sweeping mechanism. Inside swept source, the frequency sweep is driven by tunable filtering, cavity-length tuning or chromatic dispersion dynamics. It can be enabled by a mechanical motion, electric current modulation or a passive dispersive element. For swept sources based on electric tuning or dispersion dynamics, relatively stable operation is expected. But the most widely used types of swept sources have internal mechanical motions and inevitably produce operational instability. For such a swept source, the instable sweep characteristic is one of major sources of phase instability observed in final OCT data.

### Development of Oxidation Process Through Lateral Holes

In one embodiment, the N-BDR consists of only a few pairs of N-doped GaAs and AlAs material. The AlAs layers are designed in order to compensate the shrinkage of this material after the wet oxidation process. The Al content in the oxide layer is only 98% and the N-DBR is 100%. The thermal wet oxidation rate is faster on layers with high Al composition. The wet oxidation of the B-DBR is performed at the same time as the oxide layer to create the confinement current aperture. The N2 gas penetrate into all AlGaAs layers in the structure laterally at a temp of ~450 deg C including the 98% oxide layer and the N-DBR thought the holes. Targeting a given oxide aperture size for the oxide layer, the AlAs N-DBR layers are already fully oxidized. The Al as material after thermal wet oxidation is turned into AlOx which is in insulator.

### Control of Hole Depth

In one embodiment, the etching process is well controlled in order to keep the tight tolerance using different metrology tools.

### Design of Epitaxial Layer Thickness/Composition

In one embodiment, the epitaxial layer thickness and composition are designed based on the operating wavelength in order to get a high-power device across a wider tuning range with a reasonable laser voltage. The structure consists of a VCSEL body and a mechanical part.

The VCSEL body has a bottom DBR and a cavity. The DBR is a few pairs of GaAs and AlAs material, n-doped quarter lambda thick. The most top layer is highly doped than the rest of the DBR layers in order to serve as the n-contact.

A~1-2 Lambda cavity consists of a n-spacer, an active and a p-spacer. The active region has a few QWs in GaAs based material, designed to hit enough photo-luminescence (PL) gain with an offset of ~10-30 nm. The p-spacer includes a ~30 nm oxide layer of 98% AlGaAs designed to be at the node of the E-field of the structure and a short loop of a top mirror in GaAs and AlGaAs, quarter lambda thick with a highly p-doped contact layer on the top to drive the laser diode.

The mechanical part consists of a n-doped HCG layer, a thin highly doped contact layer on the top of the HCG to drive the mechanical part and a sacrificial layer (SL) that will be removed during the fab process to allow the HCG bending when applying the voltage to the MEMs. There are etch stop layers in the VCSEL structure, easy to control the wet etch process. The HCG and the SAC are GaAs based material with ~300 nm and ~1-2 Lambda thicknesses respectively. The whole epitaxial structure is grown by MOCVD with less stringent growth requirement than the thick DBR top mirror structures. This requires only one epi growth. The advantage of the HCG is the thickness, more light-weight and has a well-controlled orthogonal polarization.

In one embodiment, the final thickness of oxide layer is different than epitaxial growth of AlGaAs and includes the following:

In one embodiment, a higher Al content on DBR is higher than in the aperture. The N-DBR has 100%Al material compared to the oxide layer which only has 98%. The thermal wet oxidation rate of 100% Al is faster than the 98% Al content.

### Design of Electrical Contact Layers for Flow of Electrical Current

In one embodiment, the structure has in total 3 electrical contacts, 2 to drive the laser diode and 1 contact to drive the mechanical part. The 2 contacts to drive the laser diode are placed on the top of the VCSEL body for the p-contact and the n-contact is on the top of the N-DBR after etching the mesa. The N-contact covers the lateral holes and a part of the area surrounding the mesa. The contact to drive the HCG is deposited on the most top layer of the structure. The metal alloys used for these contacts are commonly used in the semiconductor laser fabrication to ensure a good ohmic resistance.

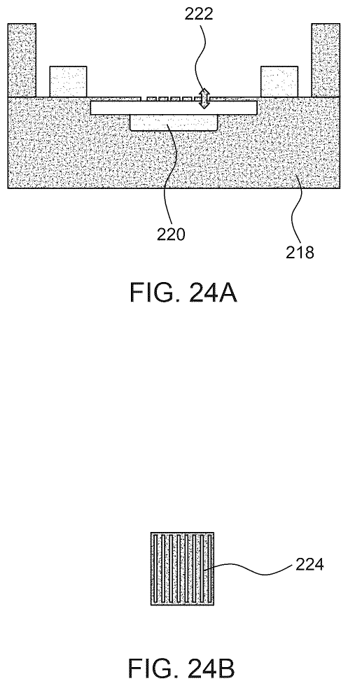

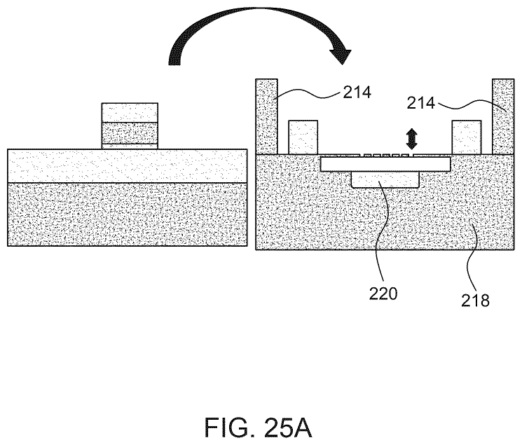

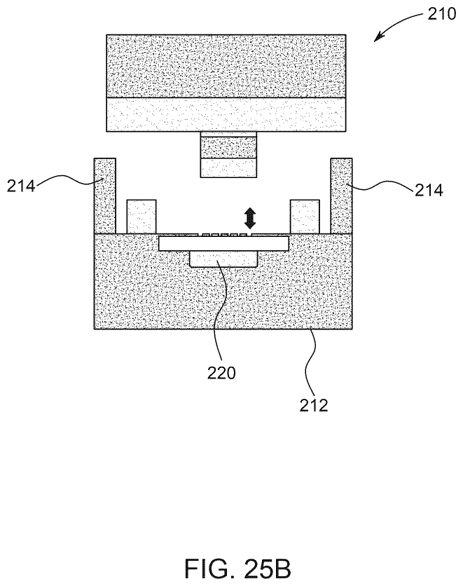

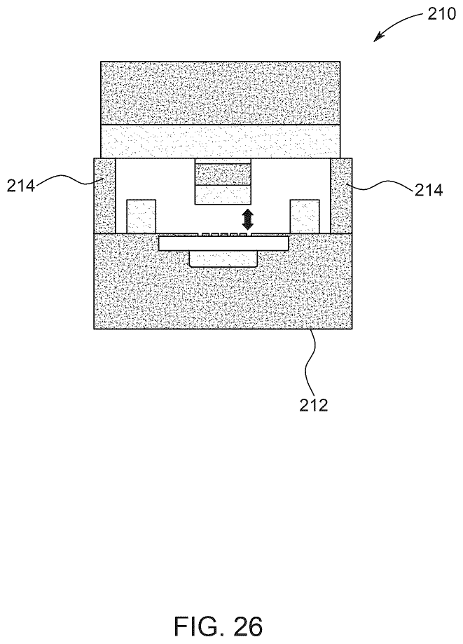

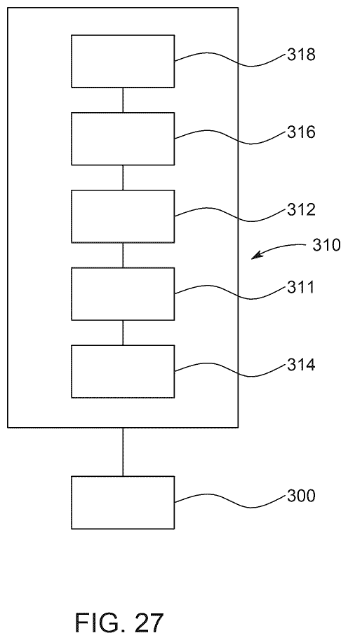

As illustrated in FIGS. 24-28 semiconductor optical amplifier 200 can use artificial intelligence (AI) for SOA 200 current operation, future operations, changes in operations, and the like, as set forth in the parameters above. Examples of AI used include but are not limited to: including linear regression, logistic regression, decision tree, SVM algorithm, Naive Bayes algorithm, KNN algorithm, K-means, random forest algorithm, dimensionality reduction, and the like.

As a non-limiting example, a k-means algorithm is used.

In various embodiments, semiconductor optical amplifier 200 can use artificial intelligence (AI), for semiconductor optical amplifier 200 operations, future operations, changes in operations, as set forth in the parameters above. Examples of AI used include but are not limited to including linear regression, logistic regression, decision tree, SVM algorithm, Naive Bayes algorithm, KNN algorithm, K-means, random forest algorithm, dimensionality reduction, and the like.

As a non-limiting example, a k-means algorithm is used.

Referring to FIGS. 24-27, in one embodiment, a user can seek from system a recommendation from the server and/or a recommendation engine 300. In one embodiment, the recommendation engine 300 make one or more recommendations that can be can be displayed at a display 302.

The recommendation engine 300 contains identifications and profiles of users who have posted recommendations/ratings, as well as profiles for users and usage feedback for videos and streamed media. A user seeking to use the recommendation engine 300 is presented (at some time) with a set of questions or the system otherwise obtains data inputs defining the characteristics of the user. In this case, the user characteristics generally define the context which is used to interpret or modify the basic goal of the user, and therefore the reference-user(s) for the user, though the user may also define or modify the context at the time of use. Various considerations are used in a cluster analysis, in which recommendations relevant to the contexts may be presented, with a ranking according to the distance function from the “cluster definition.” As discussed above, once the clustering is determined, advertisements may be selected as appropriate for the cluster, to provide a subsidy for operation of the system, and also to provide relevant information for the user about available products.

Clustering algorithms partition data into a certain number of clusters (groups, subsets, or categories). Important considerations include feature selection or extraction (choosing distinguishing or important features, and only such features); Clustering algorithm design or selection (accuracy and precision with respect to the intended use of the classification result; feasibility and computational cost; etc.); and to the extent different from the clustering criterion, optimization algorithm design or selection.

Finding nearest neighbors can require computing the pairwise distance between all points. However, clusters and their cluster prototypes might be found more efficiently. Assuming that the clustering distance metric reasonably includes close points, and excludes far points, then the neighbor analysis may be limited to members of nearby clusters, thus reducing the complexity of the computation.

There are generally three types of clustering structures, known as partitional clustering, hierarchical clustering, and individual clusters. The most commonly discussed distinction among different types of clustering's is whether the set of clusters is nested or unnested, or in more traditional terminology, hierarchical or partitional. A partitional clustering is simply a division of the set of data objects into non-overlapping subsets (clusters) such that each data object is in exactly one subset. If the clusters have sub-clusters, then we obtain a hierarchical clustering, which is a set of nested clusters that are organized as a tree. Each node (cluster) in the tree (except for the leaf nodes) is the union of its children (sub-clusters), and the root of the tree is the cluster containing all the objects. Often, but not always, the leaves of the tree are singleton clusters of individual data objects. A hierarchical clustering can be viewed as a sequence of partitional clustering's and a partitional clustering can be obtained by taking any member of that sequence; i.e., by cutting the hierarchical tree at a particular level.

There are many situations in which a point could reasonably be placed in more than one cluster, and these situations are better addressed by non-exclusive clustering. In the most general sense, an overlapping or non-exclusive clustering is used to reflect the fact that an object can simultaneously belong to more than one group (class). A non-exclusive clustering is also often used when, for example, an object is “between” two or more clusters and could reasonably be assigned to any of these clusters. In a fuzzy clustering, every object belongs to every cluster with a membership weight. In other words, clusters are treated as fuzzy sets. Similarly, probabilistic clustering techniques compute the probability with which each point belongs to each cluster.

In many cases, a fuzzy or probabilistic clustering is converted to an exclusive clustering by assigning each object to the cluster in which its membership weight or probability is highest. Thus, the inter-cluster and intra-cluster distance function is symmetric. However, it is also possible to apply a different function to uniquely assign objects to a particular cluster.

A well-separated cluster is a set of objects in which each object is closer (or more similar) to every other object in the cluster than to any object not in the cluster. Sometimes a threshold is used to specify that all the objects in a cluster must be sufficiently close (or similar) to one another. The distance between any two points in different groups is larger than the distance between any two points within a group. Well-separated clusters do not need to be spherical, but can have any shape.

If the data is represented as a graph, where the nodes are objects and the links represent connections among objects, then a cluster can be defined as a connected component; i.e., a group of objects that are significantly connected to one another, but that have less connected to objects outside the group. This implies that each object in a contiguity-based cluster is closer to some other object in the cluster than to any point in a different cluster.

A density-based cluster is a dense region of objects that is surrounded by a region of low density. A density-based definition of a cluster is often employed when the clusters are irregular or intertwined, and when noise and outliers are present. DBSCAN is a density-based clustering algorithm that produces a partitional clustering, in which the number of clusters is automatically determined by the algorithm. Points in low-density regions are classified as noise and omitted; thus, DBSCAN does not produce a complete clustering.

A prototype-based cluster is a set of objects in which each object is closer (more similar) to the prototype that defines the cluster than to the prototype of any other cluster. For data with continuous attributes, the prototype of a cluster is often a centroid, i.e., the average (mean) of all the points in the cluster. When a centroid is not meaningful, such as when the data has categorical attributes, the prototype is often a medoid, i.e., the most representative point of a cluster. For many types of data, the prototype can be regarded as the most central point. These clusters tend to be globular. K-means is a prototype-based, partitional clustering technique that attempts to find a user-specified number of clusters (K), which are represented by their centroids. Prototype-based clustering techniques create a one-level partitioning of the data objects. There are a number of such techniques, but two of the most prominent are K-means and K-medoid. K-means defines a prototype in terms of a centroid, which is usually the mean of a group of points, and is typically applied to objects in a continuous n-dimensional space. K-medoid defines a prototype in terms of a medoid, which is the most representative point for a group of points, and can be applied to a wide range of data since it requires only a proximity measure for a pair of objects. While a centroid almost never corresponds to an actual data point, a medoid, by its definition, must be an actual data point.

In the k-means clustering technique K initial centroids are selected, the number of clusters desired. Each point in the data set is then assigned to the closest centroid, and each collection of points assigned to a centroid is a cluster. The centroid of each cluster is then updated based on the points assigned to the cluster. We iteratively assign points and update until convergence (no point changes clusters), or equivalently, until the centroids remain the same. For some combinations of proximity functions and types of centroids, K-means always converges to a solution; i.e., K-means reaches a state in which no points are shifting from one cluster to another, and hence, the centroids do not change. Because convergence tends to b asymptotic, the end condition may be set as a maximum change between iterations. Because of the possibility that the optimization results in a local minimum instead of a global minimum, errors may be maintained unless and until corrected. Therefore, a human assignment or reassignment of data points into classes, either as a constraint on the optimization, or as an initial condition, is possible.

To assign a point to the closest centroid, a proximity measure is required. Euclidean (L2) distance is often used for data points in Euclidean space, while cosine similarity may be more appropriate for documents. However, there may be several types of proximity measures that are appropriate for a given type of data. For example, Manhattan (L1) distance can be used for Euclidean data, while the Jaccard measure is often employed for documents. Usually, the similarity measures used for K-means are relatively simple since the algorithm repeatedly calculates the similarity of each point to each centroid, and thus complex distance functions incur computational complexity. The clustering may be computed as a statistical function, e.g., mean square error of the distance of each data point according to the distance function from the centroid. Note that the K-means may only find a local minimum, since the algorithm does not test each point for each possible centroid, and the starting presumptions may influence the outcome. The typical distance functions for documents include the Manhattan (L1) distance, Bregman divergence, Mahalanobis distance, squared Euclidean distance and cosine similarity.

An optimal clustering will be obtained as long as two initial centroids fall anywhere in a pair of clusters, since the centroids will redistribute themselves, one to each cluster. As the number of clusters increases, it is increasingly likely that at least one pair of clusters will have only one initial centroid, and because the pairs of clusters are further apart than clusters within a pair, the K-means algorithm will not redistribute the centroids between pairs of clusters, leading to a suboptimal local minimum. One effective approach is to take a sample of points and cluster them using a hierarchical clustering technique. K clusters are extracted from the hierarchical clustering, and the centroids of those clusters are used as the initial centroids. This approach often works well, but is practical only if the sample is relatively small, e.g., a few hundred to a few thousand (hierarchical clustering is expensive), and K is relatively small compared to the sample size. Other selection schemes are also available.

The space requirements for K-means are modest because only the data points and centroids are stored. Specifically, the storage required is O((m+K)n), where m is the number of points and n is the number of attributes. The time requirements for K-means are also modest-basically linear in the number of data points. In particular, the time required is O(I×K×m×n), where I is the number of iterations required for convergence. As mentioned, I is often small and can usually be safely bounded, as most changes typically occur in the first few iterations. Therefore, K-means is linear in m, the number of points, and is efficient as well as simple provided that K, the number of clusters, is significantly less than m.

Outliers can unduly influence the clusters, especially when a squared error criterion is used. However, in some clustering applications, the outliers should not be eliminated or discounted, as their appropriate inclusion may lead to important insights. In some cases, such as financial analysis, apparent outliers, e.g., unusually profitable investments, can be the most interesting points.

Hierarchical clustering techniques are a second important category of clustering methods. There are two basic approaches for generating a hierarchical clustering: Agglomerative and divisive. Agglomerative clustering merges close clusters in an initially high dimensionality space, while divisive splits large clusters. Agglomerative clustering relies upon a cluster distance, as opposed to an object distance. For example, the distance between centroids or medoids of the clusters, the closest points in two clusters, the further points in two clusters, or some average distance metric. Ward's method measures the proximity between two clusters in terms of the increase in the sum of the squares of the errors that results from merging the two clusters.

Agglomerative Hierarchical Clustering refers to clustering techniques that produce a hierarchical clustering by starting with each point as a singleton cluster and then repeatedly merging the two closest clusters until a single, all-encompassing cluster remains. Agglomerative hierarchical clustering cannot be viewed as globally optimizing an objective function. Instead, agglomerative hierarchical clustering techniques use various criteria to decide locally, at each step, which clusters should be merged (or split for divisive approaches). This approach yields clustering algorithms that avoid the difficulty of attempting to solve a hard combinatorial optimization problem. Furthermore, such approaches do not have problems with local minima or difficulties in choosing initial points. Of course, the time complexity of O(m2 log m) and the space complexity of O(m2) are prohibitive in many cases. Agglomerative hierarchical clustering algorithms tend to make good local decisions about combining two clusters since they can use information about the pair-wise similarity of all points. However, once a decision is made to merge two clusters, it cannot be undone at a later time. This approach prevents a local optimization criterion from becoming a global optimization criterion.

In supervised classification, the evaluation of the resulting classification model is an integral part of the process of developing a classification model. Being able to distinguish whether there is non-random structure in the data is an important aspect of cluster validation.

In one embodiment, a k-means algorithm is used as follows:

The K Means Clustering algorithm finds observations in a dataset that are like each other and places them in a set. The process starts by randomly assigning each data point to an initial group and calculating the centroid for each one. A centroid is the center of the group. Note that some forms of the procedure allow you to specify the initial sets.

Then the algorithm continues as follows: it evaluates each observation, assigning it to the closest cluster. The definition of “closest” is that the Euclidean distance between a data point and a group's centroid is shorter than the distances to the other centroids.

When a cluster gains or loses a data point, the K means clustering algorithm recalculates its centroid.

The algorithm repeats until it can no longer assign data points to a closer set.

When the K means clustering algorithm finishes, all groups have the minimum within-cluster variance, which keeps them as small as possible. Sets with minimum variance and size have data points that are as similar as possible. There is variability amongst the characteristics in each cluster, but the algorithm minimizes it.

In short, the observations within a set should share characteristics. In some cases, the analysts might need to specify different numbers of groups to determine which value of K produces the most useful results.

In one embodiment, recommendation engine 300 is used to predict what will happen; or prescriptive, relative to semiconductor optical amplifier 200 data uses the data to make suggestions about what action to take, As a nonlimiting example, artificial intelligence provides predictive information about the SOA, including but not limited to temperature, modification of temperature, wavelength sweeping, wavelength, isolation, feedback, and the like.

As a non-limiting example, the artificial intelligence engine 300 is used for systems with a deep learning networks with many layers. The layered network can process extensive amounts of data and determine the “weight” of each link in the network—for example, in an image recognition system, some layers of the neural network might detect individual features of a face, like eyes, nose, or mouth, while another layer would be able to tell whether those features appear in a way that indicates a face.

As will be appreciated by those skilled in the art, there are many different artificial intelligence engines 300 that can be trained to generate suitable output values for a range of input values; the neuro-fuzzy logic engine 300 is merely one embodiment.

The measurement data, the information feeds, and the output parameters may be used to train an artificial intelligence engine 300 to control the one or more devices in response to the measurement data and information feeds In one embodiment, artificial intelligence engines 1000 can be trained to recognize temporal patterns.

In one embodiment, the measurement data, the information feeds, and the output parameters may be used to train an artificial intelligence engine 1000 to control the one or more devices in response to the measurement data and information feeds.

In one embodiment, a computing system 310 is used and includes a logic subsystem 311 and a storage subsystem 312. Computing system 310 may further include an input subsystem 314, an output subsystem 316, a communication subsystem 318, and/or other components.

Logic subsystem 311 may include one or more physical logic devices configured to execute instructions. For example, the logic subsystem may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic subsystem 311 may include one or more processors (as an example of physical logic devices) configured to execute software instructions. Additionally, or alternatively, the logic subsystem may include one or more hardware and/or firmware logic machines (as an example of physical logic devices) configured to execute hardware or firmware instructions. Processors of the logic subsystem may be single-core or multi-core, and the instructions executed thereon may be configured for sequential, parallel, and/or distributed processing. Individual components of the logic subsystem may be distributed among two or more separate devices, which may be remotely located and/or configured for coordinated processing. Aspects of the logic subsystem may be virtualized and executed by remotely accessible, networked computing devices configured in a cloud-computing configuration.

Storage subsystem 312 includes one or more physical, non-transitory memory devices configured to hold instructions executable by the logic subsystem in non-transitory form, to implement the methods and processes described herein. When such methods and processes are implemented, the state of storage subsystem12 may be transformed. e.g., to hold different data. Storage subsystem 312 may include removable and/or built-in devices. Storage subsystem 312 may include optical memory devices, semiconductor memory devices, and/or magnetic memory devices, among other suitable forms. Storage subsystem 312 may include volatile, nonvolatile, dynamic, static, read/write, read-only, random-access, sequential-access, location-addressable, file-addressable, and/or content-addressable devices. Aspects of logic subsystem 310 and storage subsystem 312 may be integrated together into one or more hardware-logic components. While storage subsystem 312 includes one or more physical devices, aspects of the instructions described herein alternatively may be propagated by a communication medium (e.g., an electromagnetic signal, an optical signal, etc.) that is not necessarily held by a physical device for a finite duration.

The terms “module,” “program,” and “engine” may be used to describe an aspect of computing system 310 implemented to perform a particular function. In some cases, a module, program, or engine may be instantiated via logic subsystem 110 executing instructions held by storage subsystem 312. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc. A “service,” as used herein, may refer to an application program executable across multiple user sessions. A service may be available to one or more system components, programs, and/or other services. In some implementations, a service may run on one or more server-computing devices.

In one embodiment, system 310 can be coupled to a memory 410, output device 412, a database 414, computing system 411, an input 416 and processor 41. System 310 can receive input from the one or more sensors.

As a non-limiting example, data, input from the one or more sensors, is received in a first format 416, translated into tokens 418, tokens are stored in an array 420, a determination is made whether the array contains composite concepts 422, if yes, the tokens are replaced in an array which include a composite with composite tokens 424, if not a determination is made whether token in the array are derived from root tokens 426, if yes then root tokens are added to the array 428, all token are added to the array is that are derived from array root tokens 430, is not, then a determination is made if tokens in the array have a relationship with and other tokens 432, if so the relationships are added to the array 434, if not an input array is constructed 436.

In one embodiment, the input array 420 is compared to the stored conceptual token array 438 and a dynamic constructed output is created using a selected output pattern as a starting point 440.

In one embodiment, an incomplete output pattern 442 is used to transmit an output array 444

As a non-limiting example, the input can be translated into a second format such as an integer format. The input may be broken into tokens which are each assigned a corresponding integer. The input is stored in an input array, which is a one-dimensional linear array that contains one token for each integer or item of input. In one embodiment, each token is assigned an integer value, so the token “cookie” may be assigned a value of “0056.”

System 310 can use training and a machine learning program that includes features, message words, message concepts, communication history, past behaviors, one or more subjects, message attributes, one or more senders, user data, training data, machine-learning program training, one or more trained ML programs, new data and one or more assessments.

The tokens are assigned predetermined integer values based on a database available to the system that stores integer values for all words. Various different tokens that have the same meaning, such as “cookie” and “cookies” may be assigned the same integer value.

Once the one-dimensional linear array is constructed, the system may then determine whether one or more tokens in the input array correlate to one or more predetermined criteria, such as for example, whether any one or more tokens in the array comprise a term of art or if two or more tokens in the array comprise a composite concept. This occurs when a string of words is meant to express a single idea. In one embodiment, if any one or more tokens are found to comprise a term of art or if one or more tokens are found to comprise a composite concept, they are replaced in the input array by a token representing the corresponding term of art or composite concept. In another embodiment, one or more tokens representing the term of art or composite concept may be added to the input array.

Common phrases may also be converted from a plurality of integers into a single integer. For example, the words “May I please have” may be represented by the integer values 001, 002, 003, and 004. The words, “Can you get me” may be represented by the integer 005, 006, 007, and 008. However, because these two strings mean the same thing, they may both be stored in the string database under the variable 056. The system will then convert these strings into the variable 056, which reduces the number of variables the system must work with and thus reduces processing time for analyzing the input and formulating a response.

The formula for calculating the number of linear arrays can be: (X1+1)(X2+1)(X3+1). (Xn+1) where n is the number of tokens in the array and X is the number of tokens corresponding to each root token. When applied to the example shown in FIG. 5, the input array is expanded into 192 separate linear arrays. There are 5 tokens in the array, so the formula would look as follows: (X1+1)(X2+1)(X3+1)(X4+1)(X5+1). The “X” for each token is replaced by the number of tokens corresponding to each token, so the formula would look as follows once the input array was expanded.

The various processes and elements, such as concepts, output patterns, and other databases utilized by the system can be stored in memory 410 such as, for example, a random access memory in order to speed processing of system. In other embodiments, these processes and elements may be stored on a hard disk drive, flash memory drive, read-only memory, or any other type of memory device depending on the needs of the user.

In one embodiment, the algorithms used can be encrypted with methods known in the art before being stored in memory 410 in order to prevent copying or reverse engineering. In one embodiment, the algorithms are compressed and encrypted for further protection. A number of methods of digital rights management as known in the art may also be used to prevent copying of the algorithms used to implement the present invention.

In one embodiment, an HCG tunable VCSEL is provided. Step index and regrowth is used for lateral optical confinement and increased efficiency for more demanding applications, such as high-speed modulation and coherent communication. As a non-limiting example, lateral optical confinement and increased efficiency are provided. In one embodiment, a 1550 nm VCSEL is created. The present VCSEL can be used with any specific wavelength of design and is not limited to 1550 nm.

HCG 34 can undergo deformation. The deformation can change the characteristic of HCG 34 and modify reflection and transmission. As a non-limiting example, if reflection is modified, VCSEL 10 can lose efficiency. The beam shape can be deformed if the transmission is modified. FIG. 2 show displacement from a resting position when HCG 34 is actuated. In one embodiment, actuation can be excited via thermal, piezoelectric or electrostatic transduction. In one embodiment, the most uniform deformation is at a central portion of HCG 34, is color at central portion. In one embodiment, a broad tuning range of VCSEL 10 requires a large displacement of HCG 34. In one embodiment, fast tuning of VCSEL 10 requires a small mass and a compact design.

In one embodiment of HCG 34 that can be used for fast tuning speed applications. In this embodiment, HCG 34 has a hexagonal frame with less deformation due to corners. There is a small deformation along the “y” axis, and a large deformation along the “x” axis.

HCG 34 can be used for a fast tuning speed applications. In this embodiment, HCT 34 has octagonal frame with support elements 35 on four sides. A lens like shape is provided. There is a large deformation along both the “y” and “x” axes

In one embodiment HCG 34 has two frames, one coupled to and in a surrounding relationship to the other. As a non-limiting example, the double-frame provides a broad tuning range. As a non-limiting example, deformation occurs primarily on the outside frame. The first, square frame has a very small deformation.

As a non-limiting example, a number of quantum wells can be the same in all junctions, but not necessarily. Individual quantum well design can also be slightly different in order to broaden spectral material gain and increase tuning range.

In one embodiment, HCG 34 acts as a partial mirror. As a non-limiting example, HCG 34 can operate as a second mirror HCG 34 is positioned at the top of the vertical resonator cavity 32, following removal of at least a portion of the sacrificial layer 44. HCG 34 is positioned at the top of the vertical resonator cavity 32. As a non-limiting example, HCG 34 is positioned on a top of the mesa. in one embodiment, a plurality of support elements, anchors, 35 hold HCG 34 in place. HCG 34 then moves, typically up and down relative to cavity 32.

Two or more support elements 35 are provided. Support elements provide support for HCG 34. As a non-limiting example, support elements 35 can have dimensions of 50 microns long, one micron wide. Their thickness is determined by a growth of HCG 34 layer. Sacrificial layer 44 is defined by epitaxial growth. As a non-limiting example, HCG deformation is provided under different points of operation. For broad tuning, HCG 34 deforms and moves away from cavity 32. A period and duty cycle of HCG 34 are determined by the amount of deformation. As a non-limiting example, a thickness of support elements 35 define their function. A modification of support elements 35 occurs when there is an actuation. This causes support elements 35 to bend and HCG 34 deforms. In one embodiment, the support elements 45 are not straight beams. In various embodiments, there can be variations of support elements 35 to modify HCG 34 deformation.

A good portion of superficial layer 44 is substantially removed, at least a portion creating anchors for HCG 34.

As a non-limiting example, a main actuation of HCG 34 can include but not be limited to: electrooptic, with an application of voltage across the surface of HCG 34, thermal, piezoelectric and the like. an air gap is between the top surface of vcsel laser 10 and HCG 34. Top DBR is not necessary.

When a voltage is applied, anchors do not allow current to flow over and cause HCG 34 to move. This creates charging between HCG 34 and the mesa 122. When voltage is increased, an attraction vis electro static actuation can be increased. This provides tuning that depends of the wavelength and power. In one embodiment, tuning up to 100 nm is provided.

In one embodiment, electrostatic actuation is used by application of a voltage

As a non-limiting example, less than 1% of the output beam is emitted from vcsel laser 10.

The remaining portions of sacrificial layer 44 not removed provide anchoring points for HCG 34. As a non-limiting example, portions of the sacrificial layer 44 remain to provide anchoring in order for HCG 34 to move.

There can be two or more support elements 35 coupled to a remaining portion of sacrificial layer 44. In one embodiment, four support elements 35 are provided. Different directions of moving HCG 34 are provided. In various embodiments, HCG 34 can have a variety of different geometries, including but not limited to hexagonal, octagonal, a double frame design and the like.

In one embodiment, the output 38 of the VCSEL laser has a long wavelength, including but not limited to from 1 micron to 1.7 microns, and in one instance, 1.365 microns.

In one embodiment, the output 38 of the VCSEL laser is a long wavelength, at least partially created from indium phosphide structure or material in the VCSEL laser 10. As a non-limiting example, VCSEL laser 10 can include an indium phosphide substrate, or a substrate that at least partially includes indium phosphide.

In one embodiment, VCSEL laser 10 is coupled to or includes a top DBR 42 or high contrast grading (HCG) 34. As a non-limiting example, bottom DBR 12 is a semiconductor DBR or a combination of a semiconductor DBR with a dielectric coating. In this manner, VCSEL 10 can include a dielectric coating. As a non-limiting example, the dielectric coating improves a broadening of a tuning range of the VCSEL laser 10.

In one embodiment, VCSEL laser 10 operates in a single mode or a multi-mode operation. As a non-limiting example, dimensions of the aperture and HCG 34 are contributing factors to the single mode operation.

In one embodiment, VCSEL laser 10 can deploy multiple tunnel junctions to enhance the output 38 of VCSEL laser 10.

As a non-limiting example, buried tunnel junctions (BTJ) improve an energy efficiency of VCSEL laser 10.

As a non-limiting example, a wavelength of the VCSEL laser output 38 can be swept to provide improved resolution. In one embodiment, the VCSEL laser output 38 is swept by modulating an HCG grating 34 up and down, e. g, its up and down movement relative to a top of VCSEL laser 10, wherein when the HCG 34 moves closes to non-extended portion relative to a top of VCSEL laser 10, a wavelength of the output 38 changes and returns closer to an original output 38 of the VCSEL laser, without extension of the grating.

In one embodiment, a mem's structure is coupled to the HCG grating 34 or top DBR 42 to create a swept source. As a non-limiting example, modulating a VCSEL laser output 38, in combination with a sweeping of wavelengths of the VCSEL laser output 38 allows for higher resolution and reduces at least a portion of atmospheric interference of VCSEL laser operation.

In one embodiment, multiple tunnel junctions increase an optical power of the VCSEL laser 19. These junctions are provided in a body of the VCSEL laser 10.

In one embodiment, a semiconductor optical amplifier is included with a modulator to allow a swept source to be modulated. As a non-limiting example, the modulation of the VCSEL laser turning the VCSEL laser on and off. As a non-limiting example, the modulation is from about 1 to 50 G. In another embodiment, the modulation is greater than 50 G.

Each of the following references is expressly incorporated herein by reference in its entirety:
Abraham, Ittai, et al. “Low-distortion inference of latent similarities from a multiplex social network.” SIAM Journal on Computing 44.3(2015 ): 617-668.
Aldenderfer, M. S., and R. K. Blashfield. Cluster Analysis. Sage Publications, Los Angeles, 1985.
Anderberg, M. R. (1973). Cluster Analysis for Applications. Academic Press, New York.
Anderson, E. (1957). A semi-graphical method for the analysis of complex problems. Proc. Nat. Acad. Sci. USA 43923-927.
Anderson, T. W. (1958). An Introduction to Multivariate Statistical Analysis. Wiley, New York.
Anderson, T. W., and Bahadur, R. R. (1962). classification into two multivariate normal distributions with different covariance matrices. Ann. Math. Statist. 33420-431.
Andrews, D. F. (1972). Plots of high-dimensional data. Biometrics 28 125-136.
Ankerst, M., M. M. Breunig, H.-P. Kriegel, and J. Sander. OPTICS: Ordering Points To Identify the Clustering Structure. In Proc. of 1999 ACM-SIGMOD Intl. Conf. on Management of Data, pages 49-60, Philadelphia, Pa., June 1999. ACM Press.
Arabie, P. (1977). clustering representations of group overlap. J. Math. Soc. 5 112-128.
Arabie, P. and Carroll, J. D. (1980). MAPCLUS: A mathematical programming approach to fitting to ADCLUS model. Psychometrika 45211-235.
Arabie, P., L. Hubert, and G. D. Soete. An overview of combinatorial data analysis. In P. Arabie, L. Hubert, and G. D. Soete, editors, Clustering and Classification, pages 188-217. World Scientific, Singapore, January 1996.
Art, D., Gnanadesikan, R., and Kettenring, J. R. (1982). Data-based metrics for cluster analysis. Utilitas Mathematica 31A 75-99.
Asimov, D. (1985). The grand tour. SLAM J. Sci. Statist. Corn-put. 6 128-143.
Auffarth, Benjamin, Yasumasa Muto, and Yasuharu Kunii. “An artificial system for visual perception in autonomous Robots.” Proceedings of the IEEE International Conference on Intelligent Engineering Systems. 2005.
Babu, B. Hari, N. Subash Chandra, and T. Venu Gopal. “Clustering Algorithms For High Dimensional Data—A Survey Of Issues And Existing Approaches.”
Baker, F. B. (1974). Stability of two hierarchical grouping techniques, Case I: Sensitivity to data errors. J. Amer. Statist. Assoc. 69440-445.
Ball, G., and D. Hall. A Clustering Technique for Summarizing Multivariate Data. Behavior Science, 12:153-155, March 1967.
Banerjee, A., S. Merugu, I. S. Dhillon, and J. Ghosh. Clustering with Bregman Divergences. In Proc. of the 2004 SIAM Intl. Conf. on Data Mining, pages 234-245, Lake Buena Vista, Fla., April 2004.
Baraglia, R., Dazzi, P., Mordacchini, M., & Ricci, L. (2013). A peer-to-peer recommender system for self-emerging user communities based on gossip overlays. Journal of Computer and System Sciences, 79(2), 291-308.
Baragliaa, R., Dazzia, P., Mordacchinib, M., & Riccic, L. A Peer-to-Peer Recommender System for self-emerging user communities based on Gossip Overlays. (2012)
Beck, Carolyn, et al. “Dynamic Coverage and Clustering: A Maximum Entropy Approach.” Distributed Decision Making and Control. Springer London, 2012. 215-243.
Becker, P. (1968). Recognitions of Patterns. Polyteknisk, Copenhagen.
Bell, P. A. and Korey, J. L. (1975). QUICLSTR: A FORTRAN program for hierarchical cluster analysis with a large number of subjects. Behavioral Research Methods and Instrumentation 7575.
Berg, Mikko. “Human abilities to perceive, understand, and manage multi-dimensional information with visualizations.” (2012).
Birkin, P. Survey Of Clustering Data Mining Techniques. Technical report, Accrue Software, San Jose, Calif., 2002.
Bhat, Sajid Yousuf, and Muhammad Abolish. “A density-based approach for mining overlapping communities from social network interactions.” Proceedings of the 2nd International Conference on Web Intelligence, Mining and Semantics. ACM, 2012.
Binder, D. A. (1978). Comment on ‘Estimating mixtures of normal distributions and switching regressions.’j Amer. Statist. Assoc. 73746-747.
Blashfield, R. K., Aldenderfer, M. S. and Morey, L. C. (1982). cluster analysis literature on validation. In Classifying Social Data. (H. Hudson, ed.) 167-176. Jossey-Bass, San Francisco.
Bock, H. H. (1985). On significance tests in cluster analysis. J. Classification 277-108.
Boley, D. Principal Direction Divisive Partitioning. Data Mining and Knowledge Discovery, 2(4): 325-344, 1998.
Bosley, Daniel, and Vivian Borst. “A General Unsupervised Clustering Tool for Unstructured Data.” matrix 100:2.
Boratto, Ludovico. “Group recommendation with automatic detection and classification of groups.” (2012).
Bradley, P. S. and U. M. Fayyad. Refining Initial Points for K-Means Clustering. In Proc. of the 15th Intl. Conf. on Machine Learning, pages 91-99, Madison, Wis., July 1998. Morgan Kaufmann Publishers Inc.
Breiman, L. Meisel, W. S., and Purcell, E. (1977). Variable kernel estimates of multivariate densities and their calibration. Technometrics 19 135-144
Brineman, L., Friedman, J. H., Olshen, R. A., and Stone, C. J. (1984). Classification and Regression Trees. Wadsworth, Belmont, Calif.
Broadbent, S. R. and Hammersley, J. M. (1957). Percolation Processes, I: Crystals and Mazes. Proc. Cambridge Philos. Soc. 53629-641.
Bu, Yingyi, et al. “The HaLoop approach to large-scale iterative data analysis.” The VLDB Journal—The International Journal on Very Large Data Bases 21.2 (2012 ): 169-190.
Buja, A., Hurify, C. and Mcdonald, J. A. (1986). A data viewer for multivariate data. Computer Science and Statistics: Proceedings of the 18th Symposium on the Interface 171-174.
Cacoullos, T. (1966). Estimation of a multivariate density. Ann. Math. Statist. 18 179-189.
Cal, Rui, et al. “Scalable music recommendation by search.” Proceedings of the 15th international conference on Multimedia. ACM, 2007.
Carrizosa, Emilio, and Dolores Romero Morales. “Supervised classification and mathematical optimization.” Computers & Operations Research 40.1 (2013 ): 150-165.
Chang, Chin-Chun, and Hsin-Yi Chen. “Semi-supervised clustering with discriminative random fields.” Pattern Recognition 45.12 (2012 ): 4402-4413.
Chen, H., Gnanadesikan, R., and Kettenring, J. R. (1974). Statistical methods for grouping corporations. Sankhya B 36 1-28.
Chen, Yen Hung. “The k Partition-Distance Problem.” Journal of Computational Biology 19.4(2012 ): 404-417.
Cheng, Hong, et al. “Clustering large attributed information networks: an efficient incremental computing approach.” Data Mining and Knowledge Discovery 25.3 (2012 ): 450-477.
Chernoff, H. (1972). The selection of effective attributes for deciding between hypotheses using linear discriminant functions. In Frontiers of Pattern Recognition. (S. Watanabe, ed.) 55-60. Academic Press, New York.
Chernoff, H. (1973a). Some measures for discriminating between normal multivariate distributions with unequal covariance matrices. In Multivariate Analysis Ill. (P. R. Krishnaiah, ed.) 337-344. Academic Press, New York.
Chernoff, H. (1973b). The use of faces to represent points in k-dimensional space graphically. J Amer. Statist. Assoc. 68 361-368.
Cherubini, Umberto, and Agnese Sironi. Bond Trading, Market Anomalies and Neural Networks: An Application with Kohonen Nets. No. _012. Society for Computational Economics.
Christou, Ioannis T., George Gekas, and Anna Kyrikou. “A classifier ensemble approach to the TV-viewer profile adaptation problem.” International Journal of Machine Learning and Cybernetics 3.4 (2012 ): 313-326.
Clunies-Ross, C. W. and Riffenburgh, R. H. (1960). Geometry and linear discrimination. Biometrika 47185-189.
Cormack, R. M. (1971). A review of classification (with discussion). J Roy. Statist. Soc. A 134321-367.
Cover, T. M. (1968). Estimation by the nearest neighbor rule. IEEE Transactions Information Theory IT-14 50-55.
Cover, T. M. and Hart, P. E. (1967). Nearest neighbor pattern classification. IEEE Transactions, Information Theory IT-13 21-27.
Dallal, G. E. (1975) A user's guide to J. A. Hartigan's clustering algorithms. (unpublished manuscript) Yale University.
Day, N. E. (1969). Estimating the components of a mixture of normal distributions. Biometrika 56463-474.
Day, N. E., and Kerridge, D. F., (1967). A general maximum likelihood discriminant. Biometrics 23313-323. 94
de Master, Trabajo Fin. “Novelty and Diversity Enhancement and Evaluation in Recommender Systems.” (2012).
Defays, D. (1977). An efficient algorithm for a complete link method. Computer Journal 20364-366.
Derrac, Joaquín, Isaac Triguero, Salvador García, and Francisco Herrera. “Integrating instance selection, instance weighting, and feature weighting for nearest neighbor classifiers by coevolutionary algorithms.”

It is to be understood that the present disclosure is not to be limited to the specific examples illustrated and that modifications and other examples are intended to be included within the scope of the appended claims. Moreover, although the foregoing description and the associated drawings describe examples of the present disclosure in the context of certain illustrative combinations of elements and/or functions, it should be appreciated that different combinations of elements and/or functions may be provided by alternative implementations without departing from the scope of the appended claims. Accordingly, parenthetical reference numerals in the appended claims are presented for illustrative purposes only and are not intended to limit the scope of the claimed subject matter to the specific examples provided in the present disclosure.

1. A light emitting apparatus, comprising:
a VCSEL system, comprising:
a tunable VCSEL laser including:
a first mirror;
one or more active regions with a first active region adjacent to the first mirror, each of an active region including quantum wells and barriers, each of an active region surrounded by one or more p-n junctions, the one or more active regions including a selected shape structure each with a tunnel junction (TJ) of a plurality of TJs;
one or more apertures provided with the selected shape structure;
one or more buried tunnel junctions (BTJ), additional TJ's, planar structures and a plurality of additional BTJ's are created during a regrowth process that is independent of a first growth process of the first mirror, the active region and the one or more TJs;
one or more oxide layers and an epitaxial growth of an AlGaAs;
one or more electrical confinement apertures defined by the one or more BTJ's, additional TJ's, planar structures and additional BTJ;
a vertical resonator cavity disposed over the electrical confinement aperture;
a high contrast grating (HCG) operating as a second mirror positioned over the vertical resonator cavity, the HCG configured to reflect a first portion of light back into the vertical resonator cavity, and a second portion of the light as an output beam from the tunable VCSEL laser, the HCG structure being layered on the selected shape structure;
wherein a shape of the output beam of the tunable VCSEL laser is determined by a geometric shape of the one or more BTJ apertures, apertures for additional TJ's, planar structures and additional BTJ's, with a transmission function of the HCG and is designed according to the desired optical transmission function of the application; and a DBR on one side of the junctions, and an HCG grading on the other side of the junctions; and
wherein he VCSEL has a final thickness of an oxide layer different than the epitaxial growth of the AlGaAs.

1. The apparatus of claim 1, wherein the VCSEL is configured to have a faster oxidation in the DBR than in an aperture.

2. The apparatus of claim 1, further wherein the VCSEL has a higher Al content on a DBR than in an aperture.

3. The apparatus of claim 1, wherein the VCSEL has only one epitaxial growth and no wafer bonding.

4. The apparatus of claim 1, wherein the VCSEL is a monolith VCSEL.

5. The apparatus of claim 1, wherein the output of the VCSEL laser has a long wavelength.

6. The apparatus of claim 1, wherein the long wavelength is from 1 micron to 1.7 microns.

7. The apparatus of claim 3, wherein the long wavelength is 1.365 microns.

8. The apparatus of claim 3, wherein the output of the VCSEL laser is a long wavelength, at least partially created from indium phosphide structure in the laser structure.

9. The apparatus of claim 4, wherein the VCSEL laser includes an indium phosphide substrate.

10. The apparatus of claim 1, wherein the VCSEL laser includes or is coupled to a top DBR or a high contrast grating (HCG).

11. The apparatus of claim 1, wherein a bottom DBR is a semiconductor DBR or a combination of a semiconductor DBR with a dielectric coating.

12. The apparatus of claim 1, wherein the VCSEL laser includes a dielectric coating.

13. The apparatus of claim 1, wherein the VCSEL laser operates in a single mode or a multi-mode operation.

14. The apparatus of claim 1, wherein the VCSEL laser operates in a single mode.

15. The apparatus of claim 10, wherein dimensions of the aperture and HCG are contributing factors to a single mode operation.

16. The apparatus of claim 13, wherein the VCSEL laser can deploy multiple tunnel junctions to enhance the output of the VCSEL laser.

17. The apparatus of claim 10.1, wherein the dielectric coating improves a broadening of a tuning range of the VCSEL laser.

18. The apparatus of claim 1, wherein buried tunnel junctions improve an energy efficiency of the VCSEL laser.
